(19) United States
(12) Patent Application Publication
(10) Pub. No.: US 2024/0360169 A1
(43) Pub. Date: Oct. 31, 2024

(54) BLUE AND NARROW BAND GREEN AND RED EMITTING METAL COMPLEXES

(71) Applicant: Arizona Board of Regents on behalf of Arizona State University, Scottsdale, AZ (US)

(72) Inventors: Jian Li, Tempe, AZ (US); Zhi-Qiang Zhu, Chandler, AZ (US)

(21) Appl. No.: 18/757,817

(22) Filed: Jun. 28, 2024

Related U.S. Application Data
(63) Continuation of application No. 16/291,395, filed on Mar. 4, 2019, now Pat. No. 12,037,348.
(60) Provisional application No. 62/640,656, filed on Mar. 9, 2018.
(60) Provisional application No. 62/640,659, filed on Mar. 9, 2018.

(51) Int. Cl. C07F 15/00; H10K 50/11; H10K 85/30; H10K 101/10

(52) U.S. Cl. CPC C07F 15/0086; H10K 85/346; H10K 50/11; H10K 2101/10

(57) ABSTRACT

The present invention includes tetradentate platinum (II) complexes for narrow band green and red phosphorescent emitters. The present invention also includes blue emitting metal complexes with six-membered chelate rings based on fused carbazole. The present invention also includes organic light emitting diodes (OLEDS) including these complexes, and devices including these OLEDS.

### CROSS REFERENCE TO RELATED APPLICATIONS

The present application claims priority to U.S. Provisional Patent Application Nos. 62/640,656, filed on Mar. 9, 2018, and 62/640,659, filed on Mar. 9, 2018, all of which are incorporated by reference herein in their entireties.

### TECHNICAL FIELD

This invention relates to blue emitting metal complexes with six-membered chelate rings based on fused carbazole, organic light-emitting diodes (OLEDs) including these complexes, and devices including these OLEDs. This invention also relates to tetradentate platinum (II) complexes for narrow band green and red phosphorescent emitters, organic light emitting diodes (OLEDS) including these complexes, and devices including these OLEDS.

### BACKGROUND

Phosphorescent cyclometalated metal complexes have attracted attention due to their potential applications as phosphorescent emitters for organic light-emitting diodes (OLEDs). Through diligent materials design, efficient OLEDs across parts of the visible spectrum have been achieved. However, the development of stable and efficient blue emitters and stable narrow band green and red emitters remains a challenge.

### SUMMARY

A series of platinum (II) complexes have been designed and synthesized. These complexes provide improved color purity and enhanced operational stability and are suitable for luminescent labels, emitters for organic light emitting diodes, and lighting applications.

### BRIEF DESCRIPTION OF THE DRAWINGS

FIG. 1A depicts a schematic energy level diagram for light-emitting complexes with carbazole skeletons. FIG. 1B depicts a schematic energy level diagram for light-emitting complexes with carbazole skeleton having fused aryl groups.

FIG. 2 depicts an organic light emitting diode (OLED).

FIG. 3 shows emission spectra of a platinum (II) complex synthesized according to Example 2.

### DETAILED DESCRIPTION

General Formulas I-VIII represent blue light emitting platinum complexes with six-membered chelate rings based on fused carbazole suitable for full color displays and lighting applications. The complexes of General Formulas I-VIII have a decreased emission spectral bandwidth at room temperature due at least in part to the introduction of fused aryls to the carbazole skeleton. As depicted, complexes of General Formulas I-VIII have an axis of symmetry through M and X, that is, between each pair of Arⁿ, where n is an integer from 1 to 4.

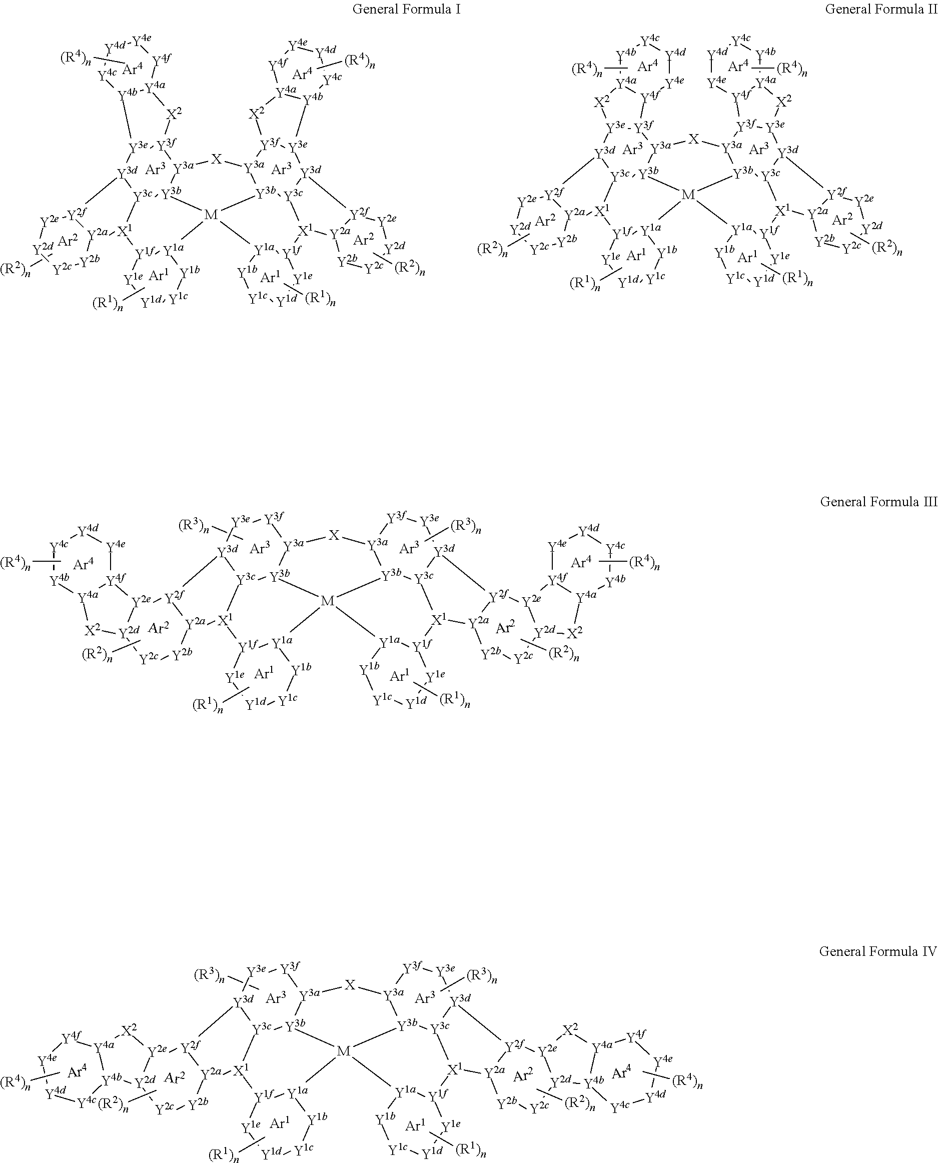

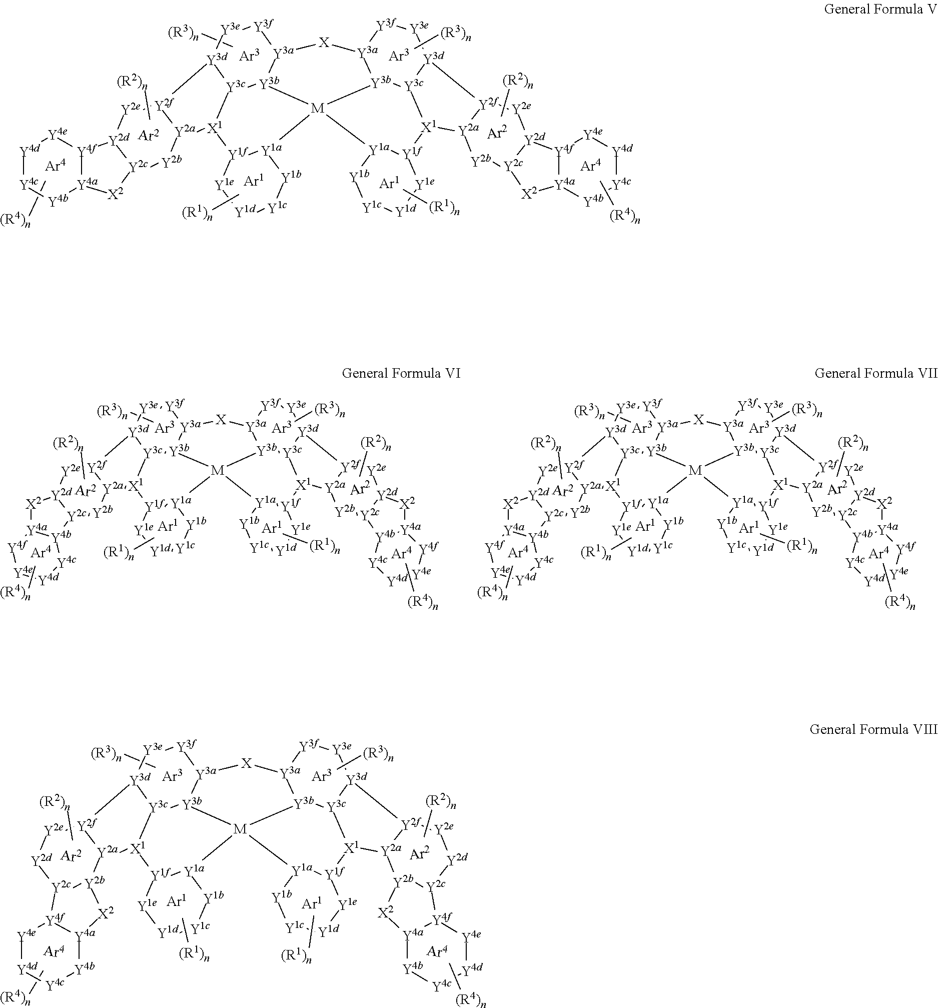

In General Formulas I-VIII:
M is Pt²⁺;
R¹, R², R³, and R⁴, if present, each independently represents hydrogen, deuterium, trideuteriummethyl, pentadeuteriumphenyl, halogen, hydroxyl, nitro, nitrile, thiol; or substituted or unsubstituted, amino, alkoxy, aryl, or C₁-C₄ alkyl;
each n independently represents an integer, valency permitting;
Y^{1a}, Y^{1b}, Y^{1c}, Y^{1d}, Y^{1e}, Y^{1f}, Y^{2a}, Y^{2b}, Y^{2c}, Y^{2d}, Y^{2e}, Y^{2f}, Y^{3a}, Y^{3b}, Y^{3c}, Y^{3d}, Y^{3e}, Y^{3f}, Y^{4a} Y^{4b}, Y^{4c}, Y^{4d}, Y^{4e}, and Y^{4f} each independently represents C, N, Si, O, or S; and
each of X, X¹ and X² is present or absent, and each X, X¹, and X² present independently represents a single bond, NR, PR, CRR′, SiRR′, CRR′, SiRR′, O, S, S═O, O═S═O, Se, Se═O, or O═Se═O, and wherein R and R′ each independently represents hydrogen, deuterium, trideuteriummethyl, pentadeuteriumphenyl, nitrile, halogen, hydroxy, nitro, thiol, or substituted or unsubstituted alkoxy, aryl, amino, or C₁-C₄ alkyl.
Ar¹, Ar², Ar³, and Ar⁴ independently represent six membered aryl and heteroaryl rings, including phenyl and pyridinyl rings.

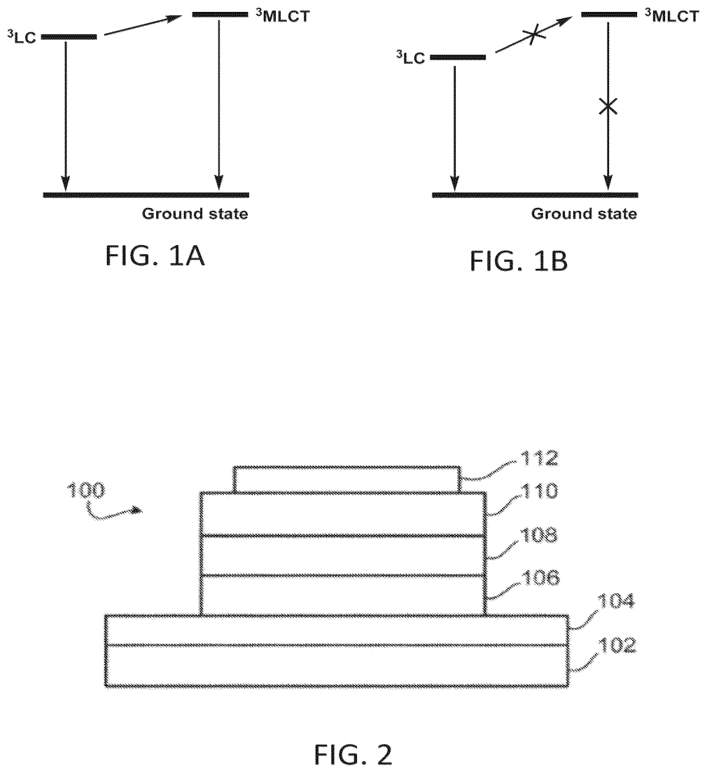

FIG. 1A depicts an energy level diagram for light emitting complexes having a carbazole skeleton, such as PtNON, depicted below.

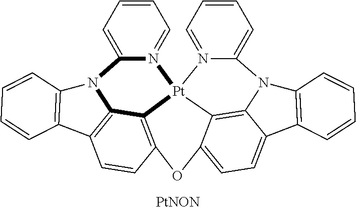

Room temperature emission spectra of these complexes are very broad. FIG. 1B depicts an energy level diagram for light-emitting complexes of General Formulas I-VIII having carbazole skeletons with fused aryl groups. One example, PtNON-S56, is depicted below.

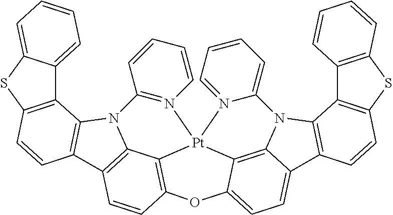

The aryls fused to the carbazole skeletons of the complexes of General Formulas I-VIII expand the conjugation system, contributing to a shift in energy levels. Compared to the energy level diagram in FIG. 1A, the energy level diagram in FIG. 1B shows a lowering of the energy of the ³LC (ligand-centered) state while the energy of the ³MLCT (metal-to-ligand charge transfer) state decreases only slightly if at all, remaining essentially the same. Thus, the energy gap between the ³LC state and the ³MLCT state at room temperature is increased due at least in part to the expanded conjugation system. It is believed that this shift increases the ³LC character of the Ti state compared to complexes with carbazole skeletons and no fused aryl groups, thereby advantageously resulting in narrower room temperature emission spectra.

Examples of compounds of General Formulas I-VIII are provided below, where each R¹ and R² are previously defined.

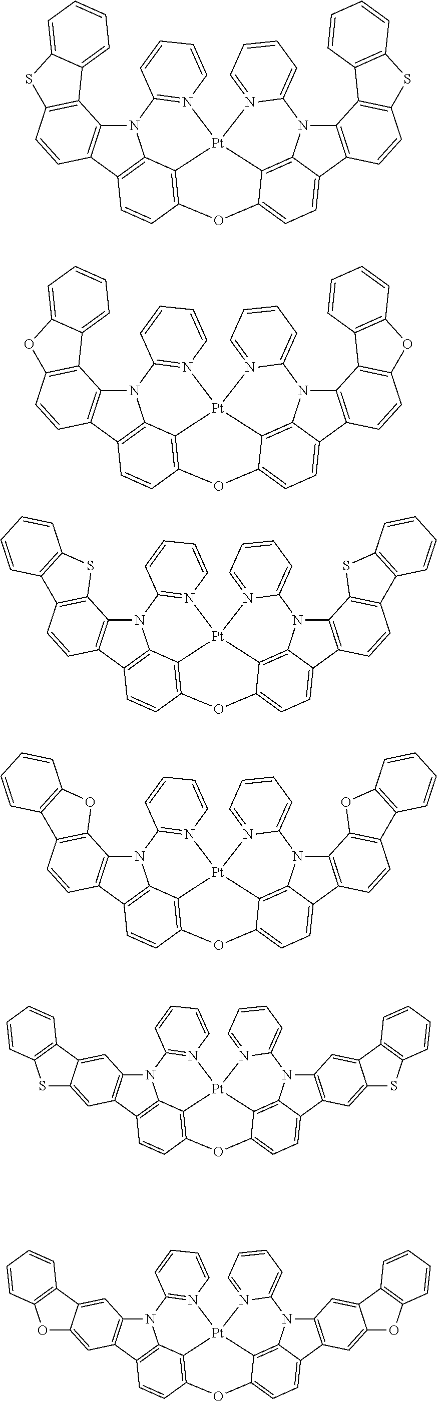

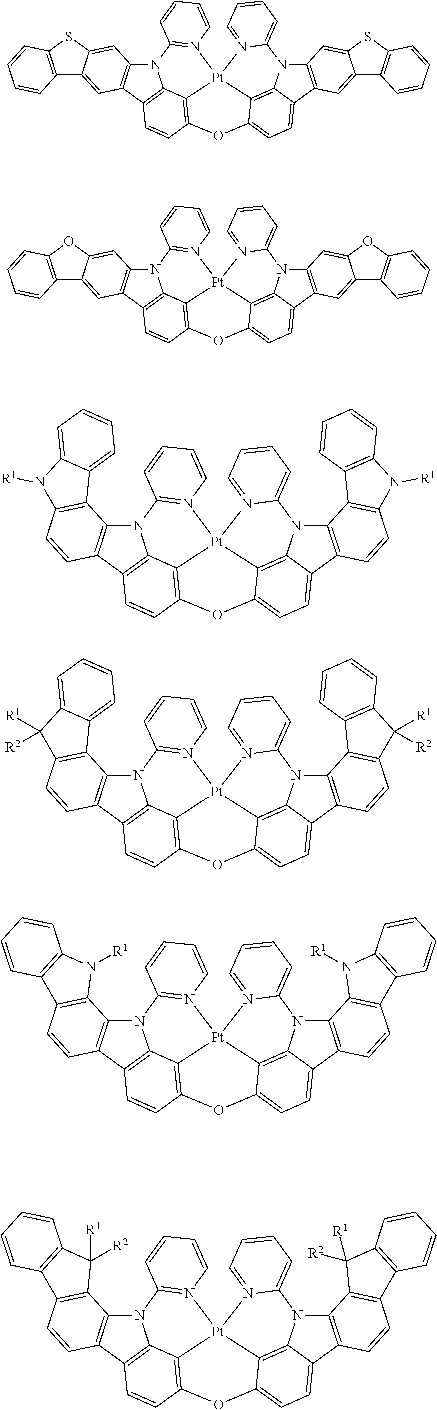

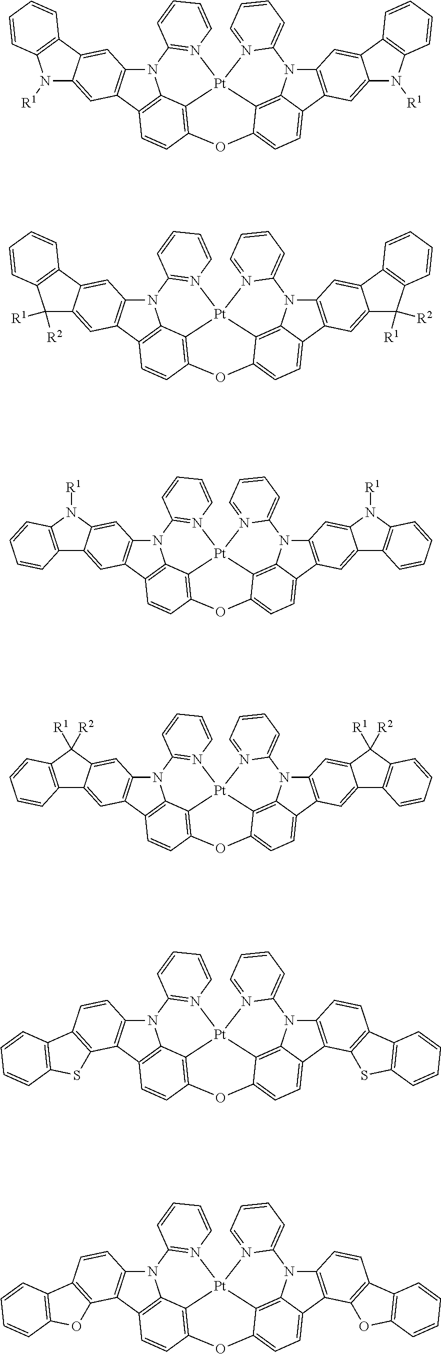

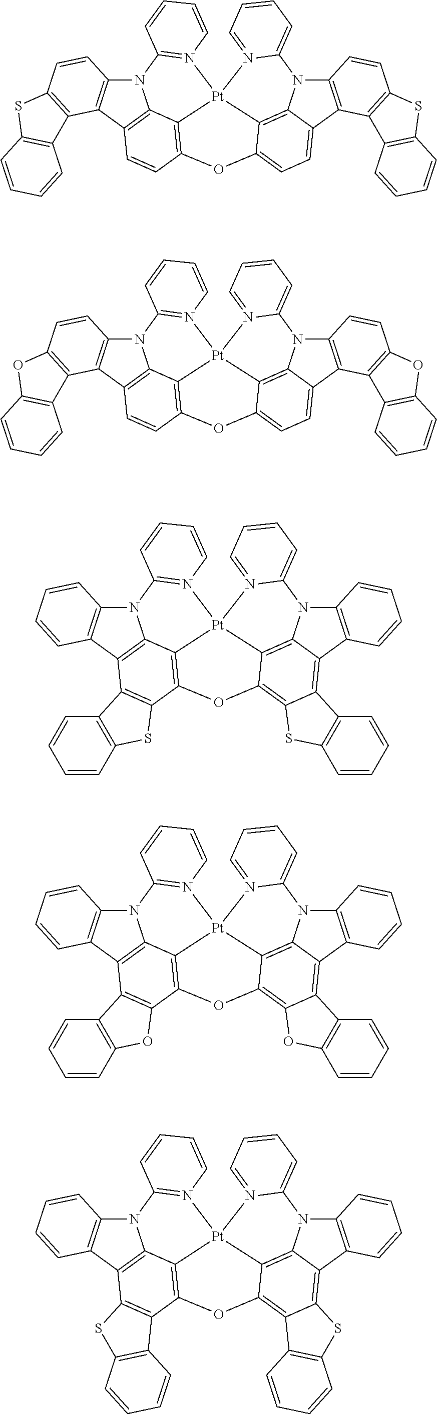

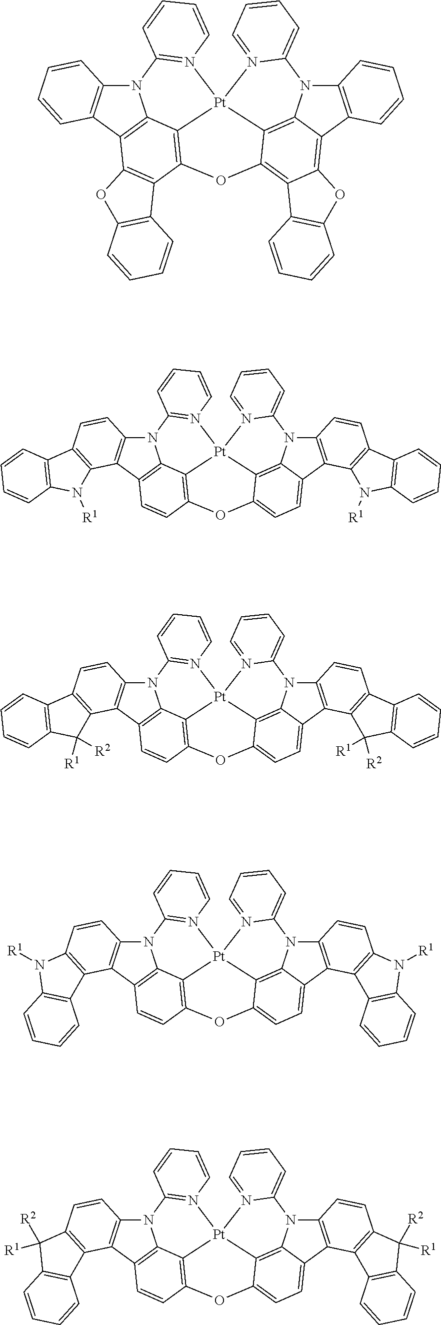

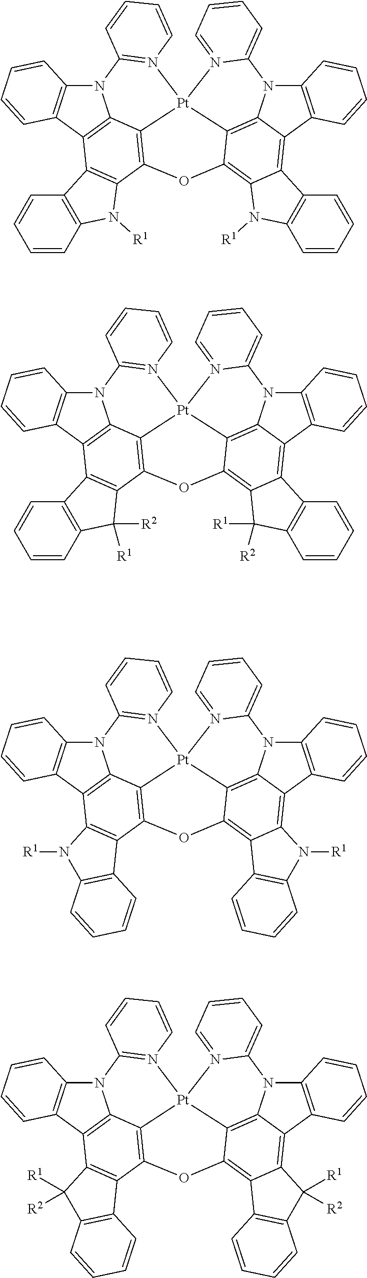

General Formula IX represents tetradentate platinum (II) complexes for narrow green and red light phosphorescent emitters suitable for full color displays and lighting applications.

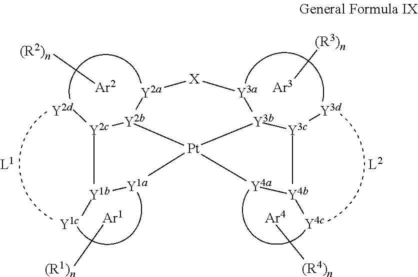

In General Formula IX:
Ar¹, Ar², Ar³, and Ar⁴ each individually represents aryl, heteroaryl, fused aryl, or fused heteraryl having 5 to 10 ring atoms;
R¹, R², R³ and R⁴ each independently represents hydrogen, deuterium, trideuteriummethyl, pentadeuteriumphenyl, halogen, hydroxyl, nitro, nitrile, thiol, or substituted or unsubstituted alkoxy, aryl, amino, or C₁-C₄ alkyl;
each n independently represents an integer, valency permitting;
Y^{1a}, Y^{1b}, Y^{1c}, Y^{2a}, Y^{2b}, Y^{2c}, Y^{2d}, Y^{3a}, Y^{3b}, Y^{3c}, Y^{3d}, Y^{4a}, Y^{4b}, and Y^{4c} each independently represents C, N, Si, O, or S;
X represents O, S, NR, CRR′, SiRR′, PR, BR, S═O, O═S═O, Se, Se═O, or O═Se═O, where each R and R′ independently represents hydrogen, deuterium, trideuteriummethyl, pentadeuteriumphenyl, hydroxyl, nitro, nitrile, thiol, or substituted or unsubstituted alkoxy, aryl, amino, or C₁-C₄ alkyl;
L¹ and L² is each independently present or absent, and if present, each independently represents an alkyl, alkoxy, alkenyl, alkynyl, hydroxy, amine, amide, thiol, aryl, heteroaryl, cycloalkyl, or heterocyclyl linking group.

Examples of suitable aryl, heteroaryl, fused aryl, or fused heteroaryl rings having 5 to 10 ring atoms for Ar¹, Ar², Ar³, and Ar⁴ include phenyl, pyridinyl, pyrimidinyl, pyrazinyl, quinolinyl, isoquinolinyl, naphthalenyl, benzimidazolyl, quinazolinyl, quinoxalinyl.

Examples of complexes of General Formula IX are shown below, where:
each Z¹, Z², Z³, and Z⁴ present independently represents C or N; and
each R⁵ and R⁶ present independently represents hydrogen, deuterium, trideuteriummethyl, pentadeuteriumphenyl, halogen, hydroxyl, nitro, nitrile, thiol, or substituted or unsubstituted alkoxy, aryl, amino, or C₁-C₄ alkyl; and
each n independently represents an integer, valency permitting.

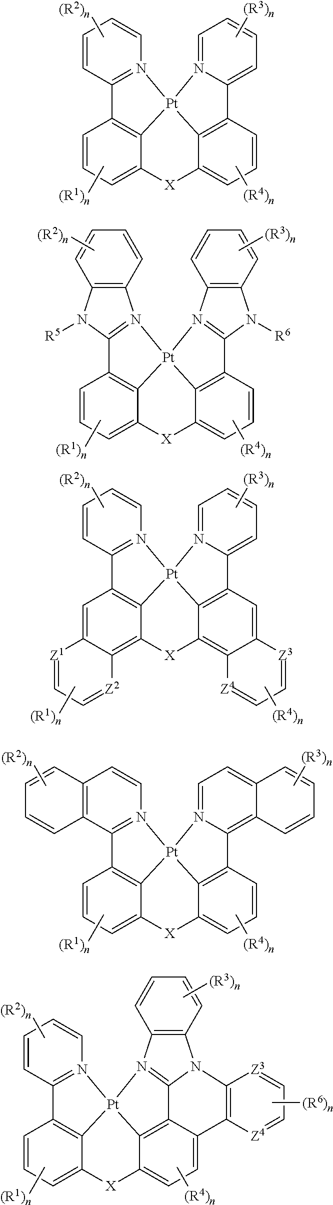

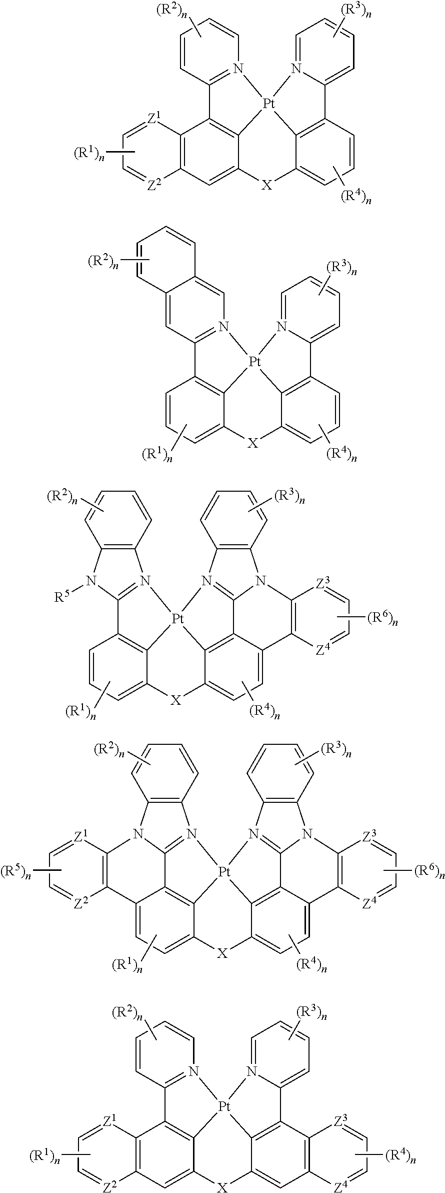

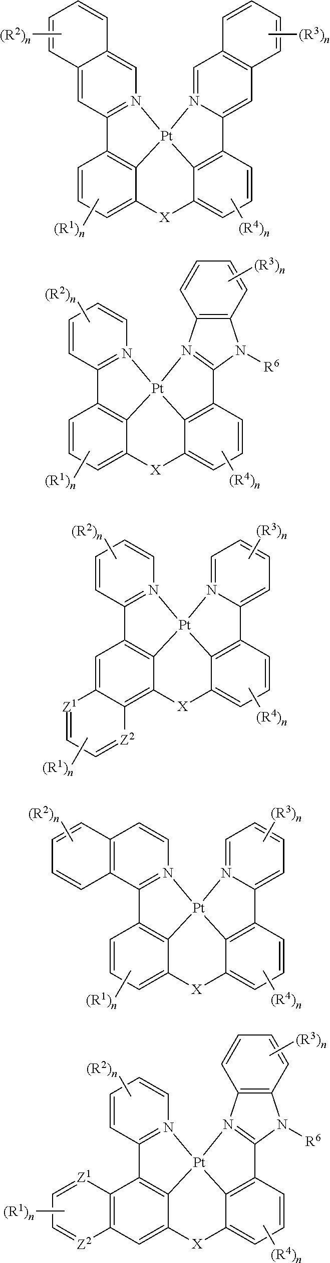

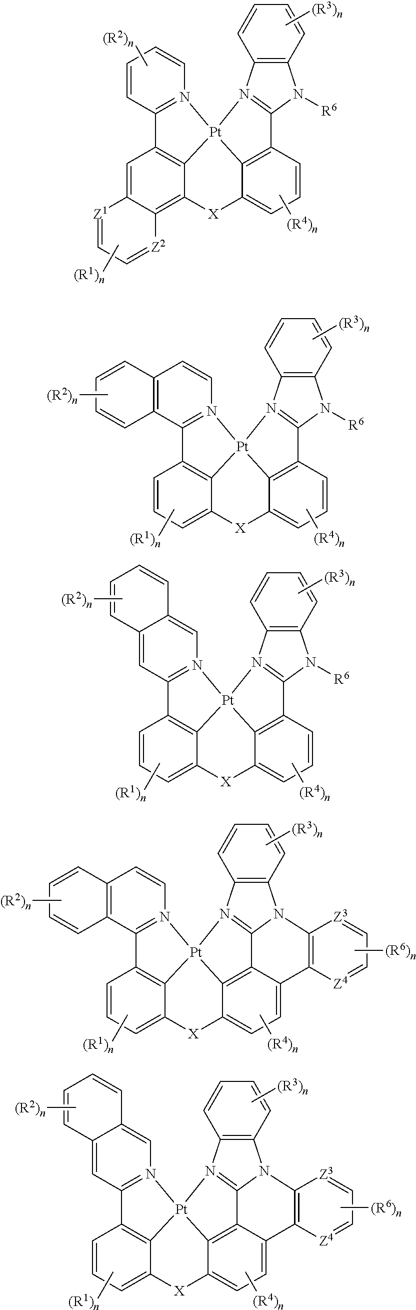

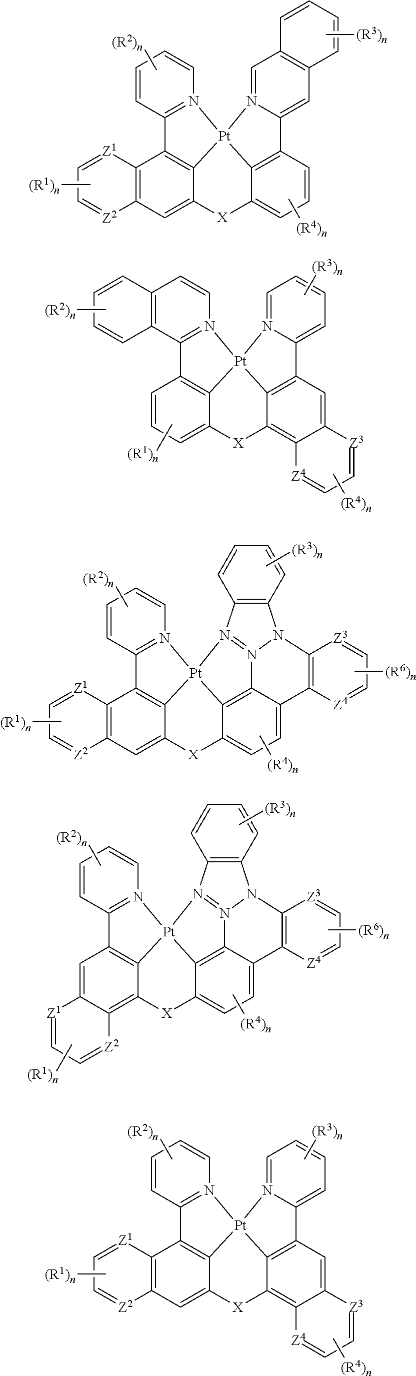

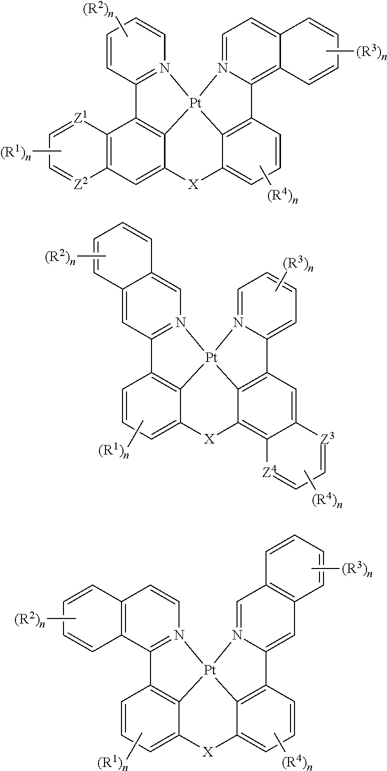

Examples of complexes of General Formula IX are shown below, where each R and R′, if present, independently represents substituted or unsubstituted alkoxy, aryl, heteroaryl, trideuteriummethyl, pentadeuteriumphenyl or C₁-C₄ alkyl.

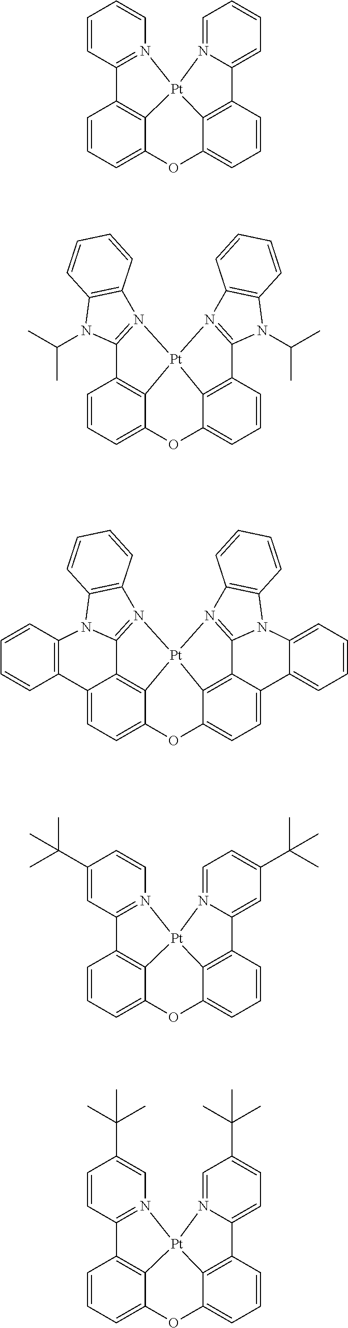

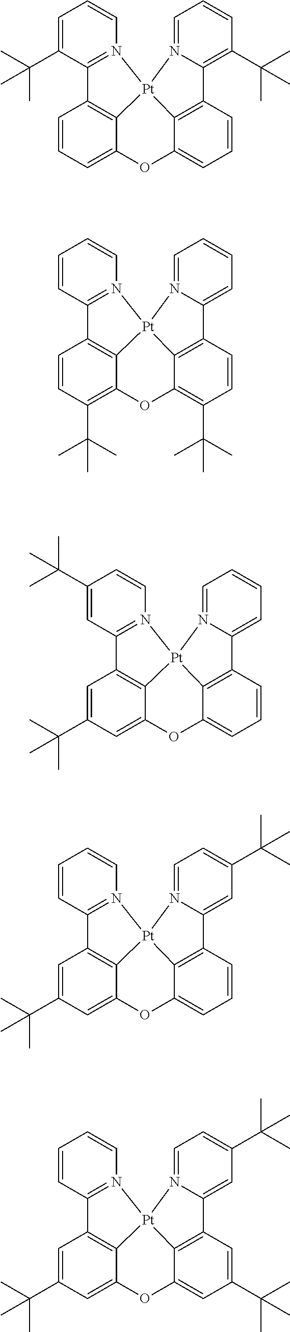

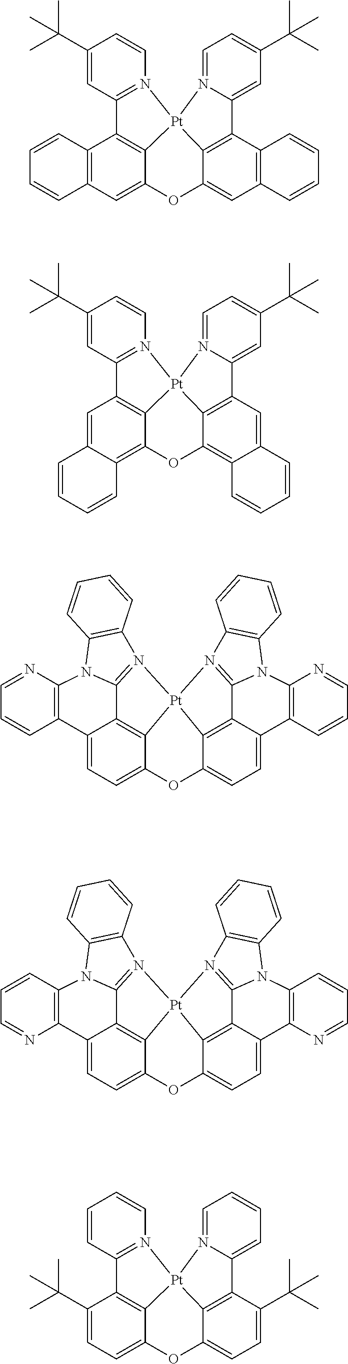

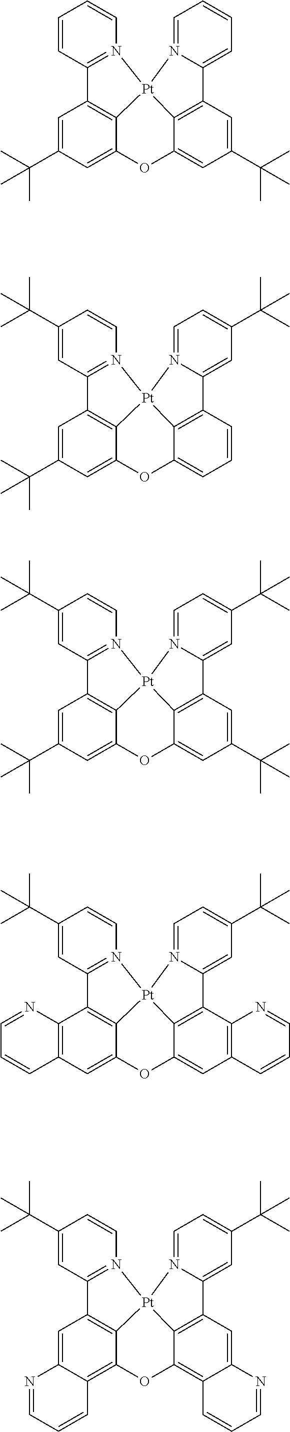

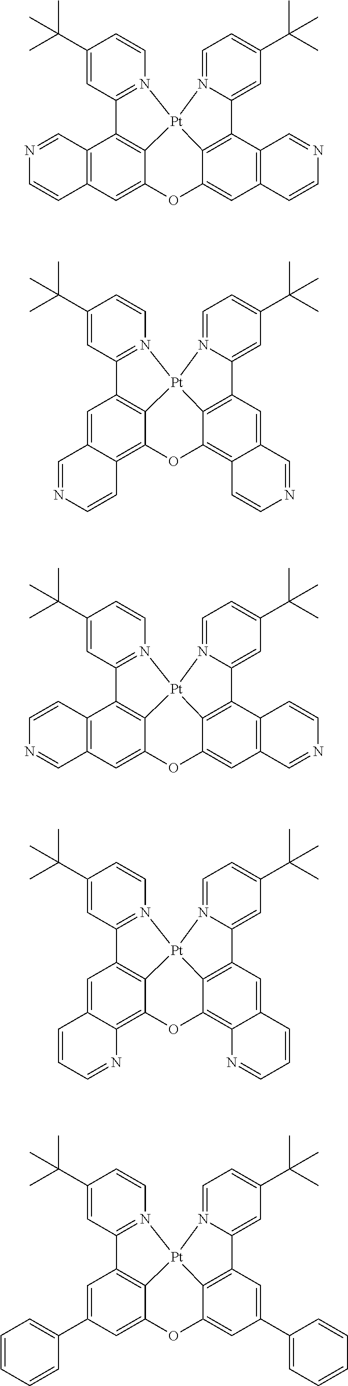

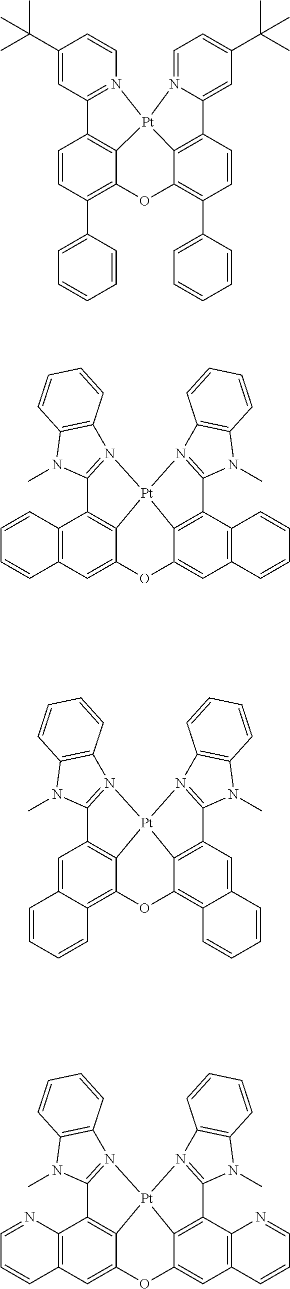

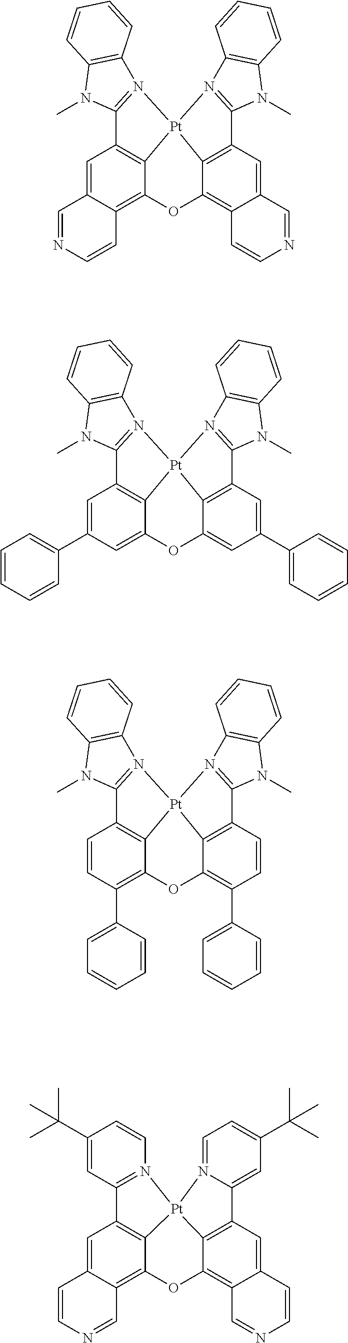

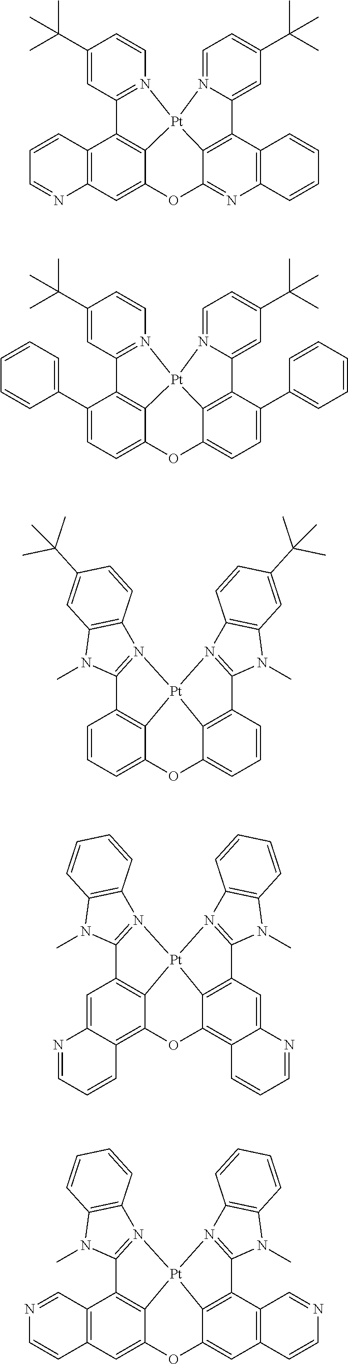

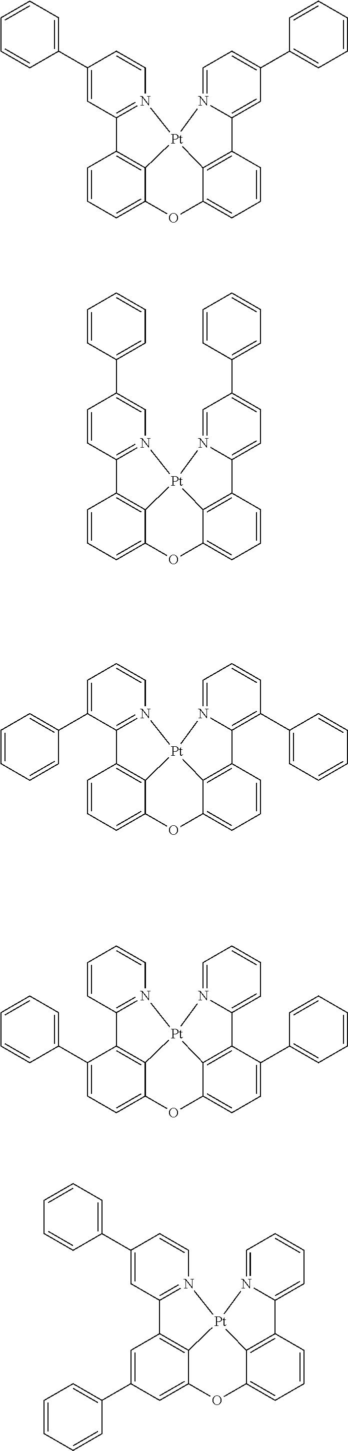

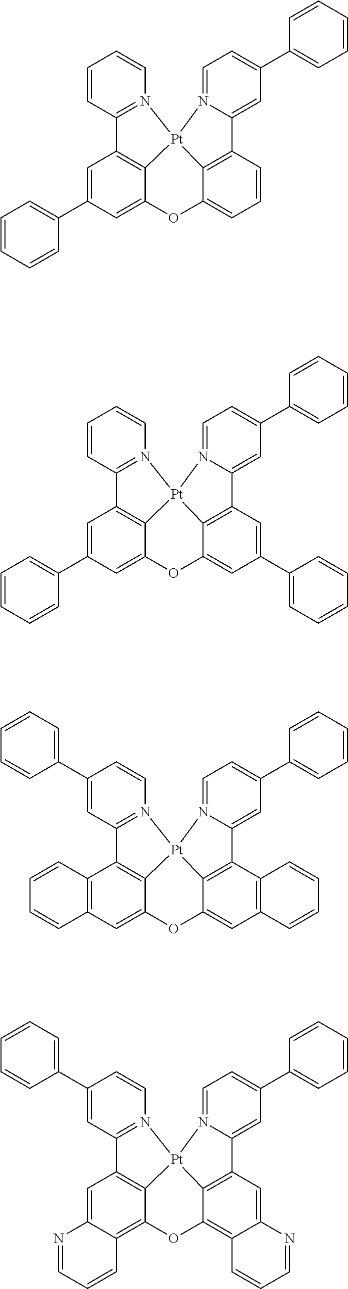

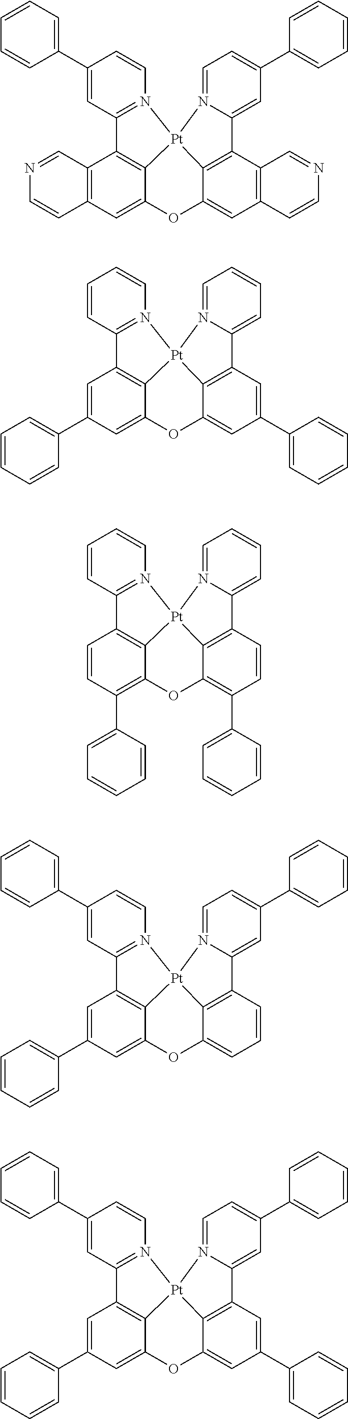

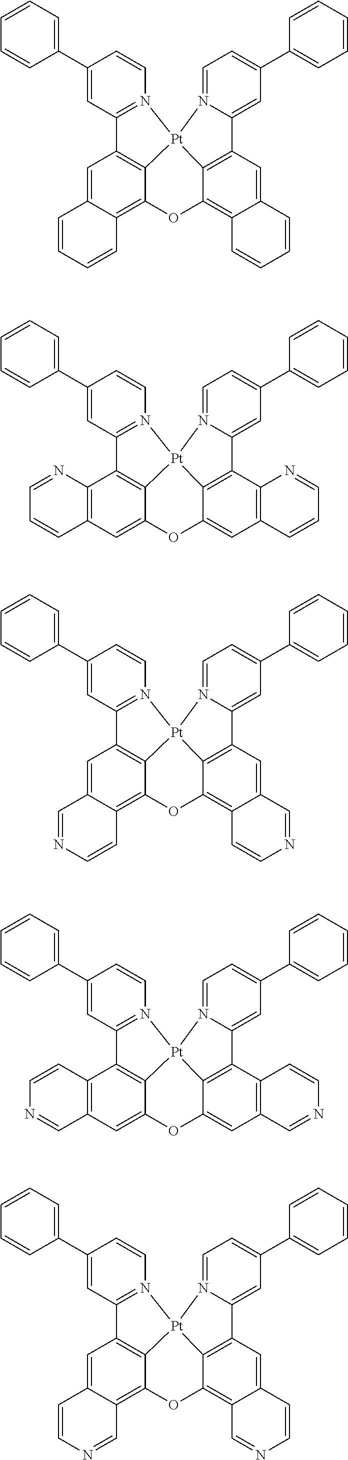

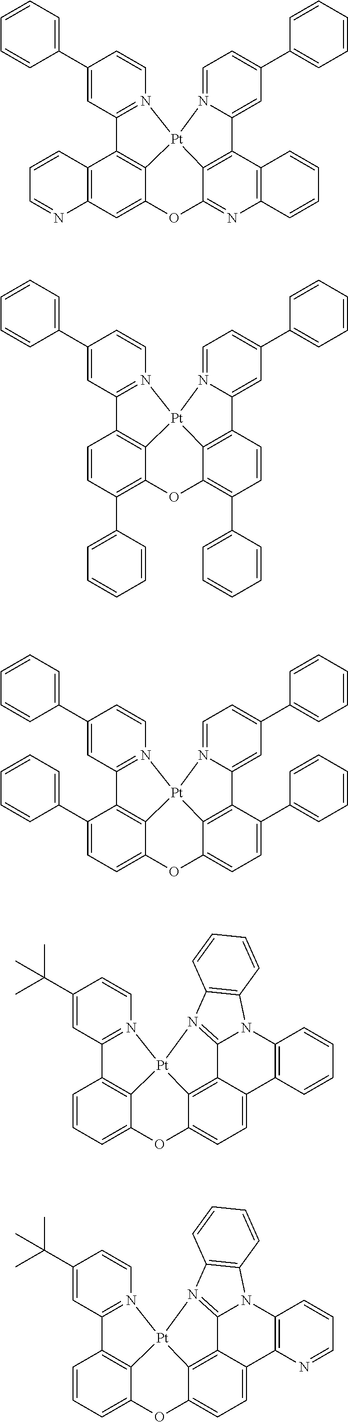

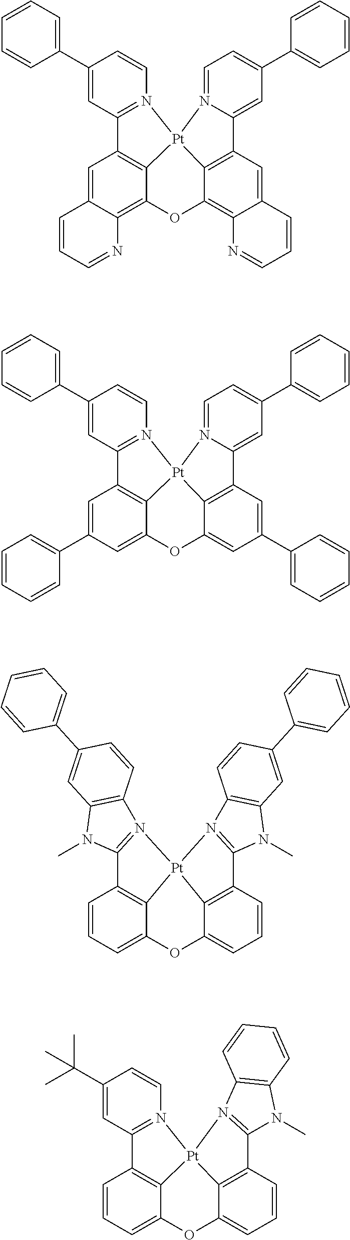

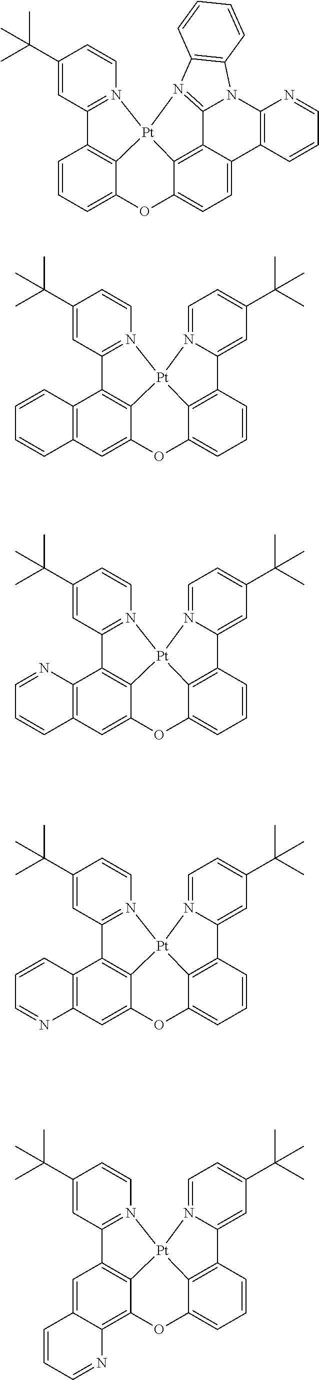

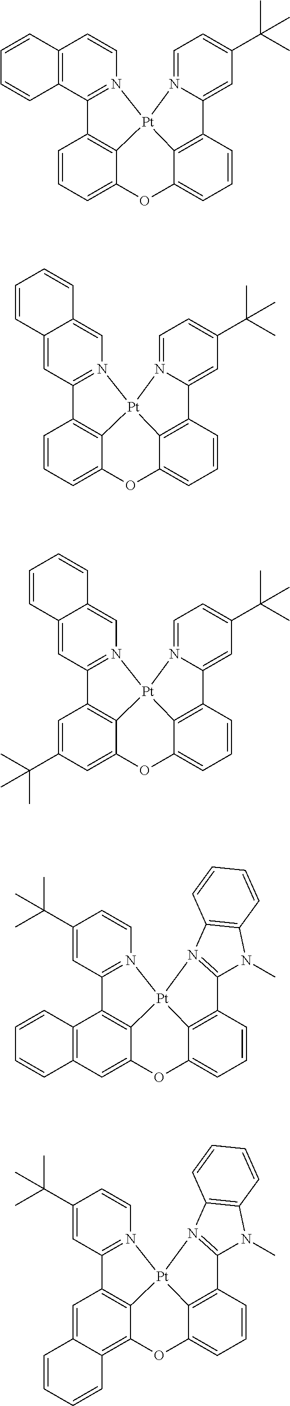

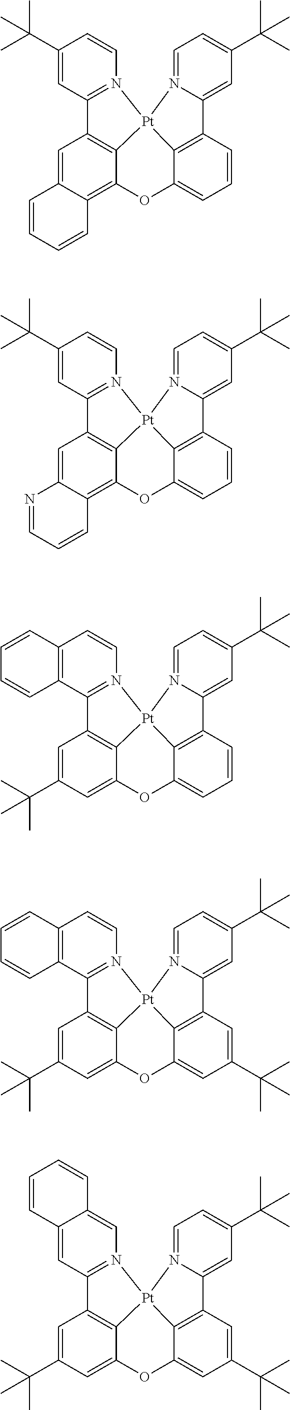

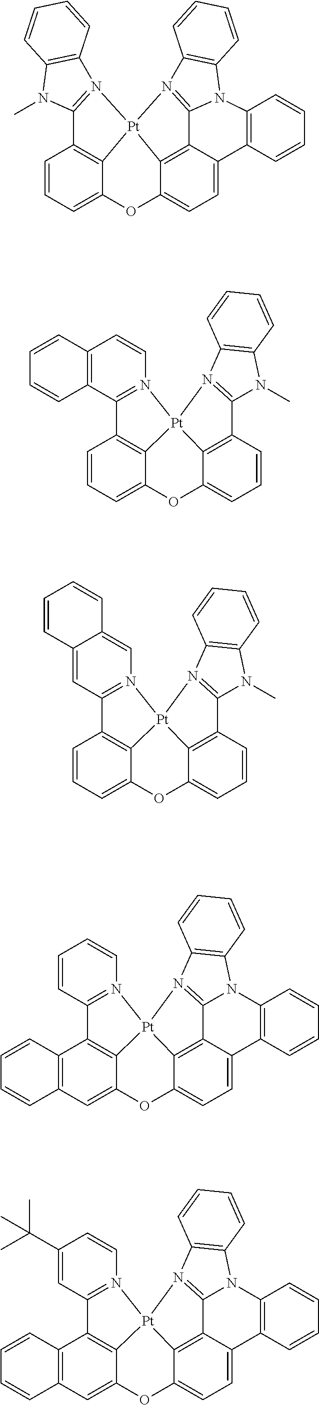

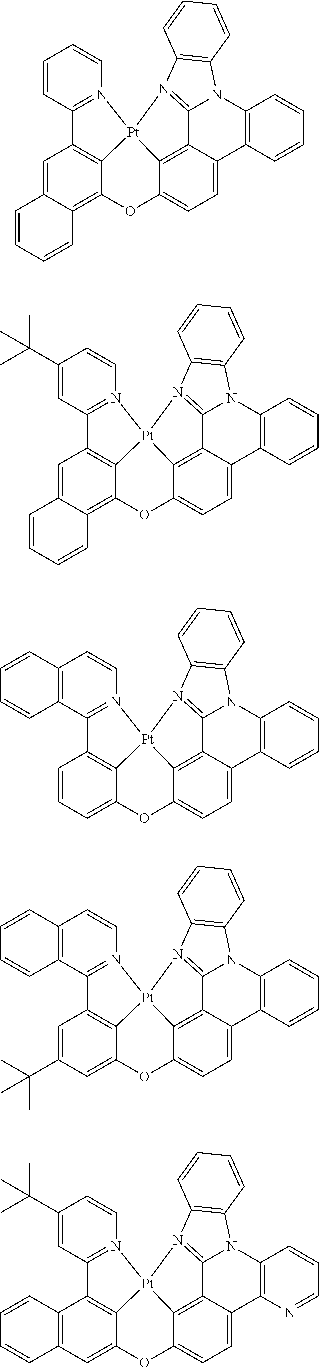

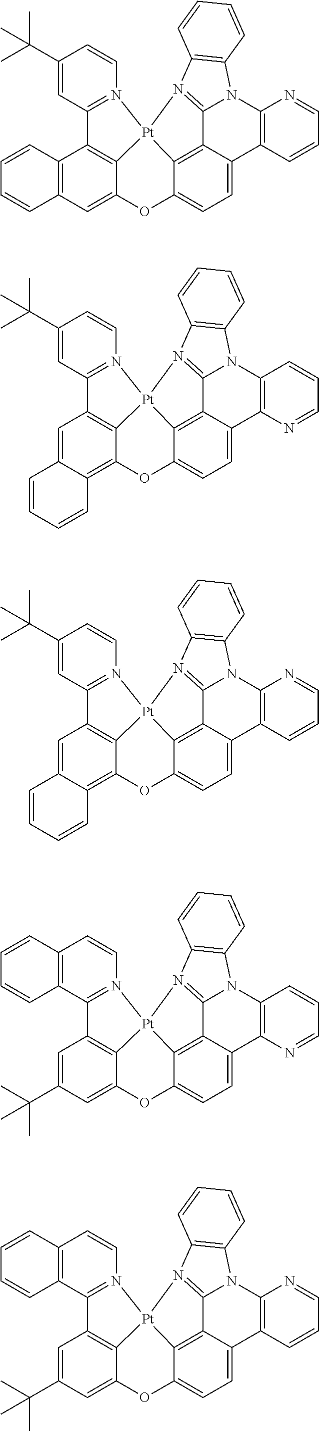

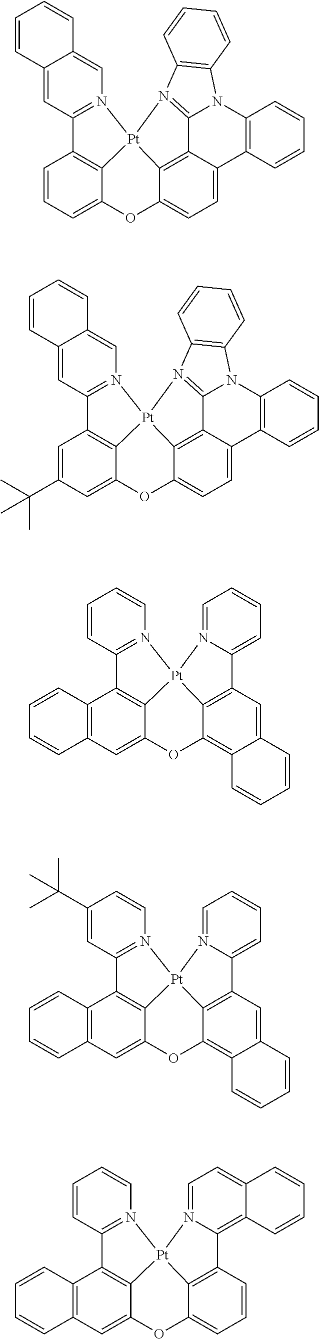

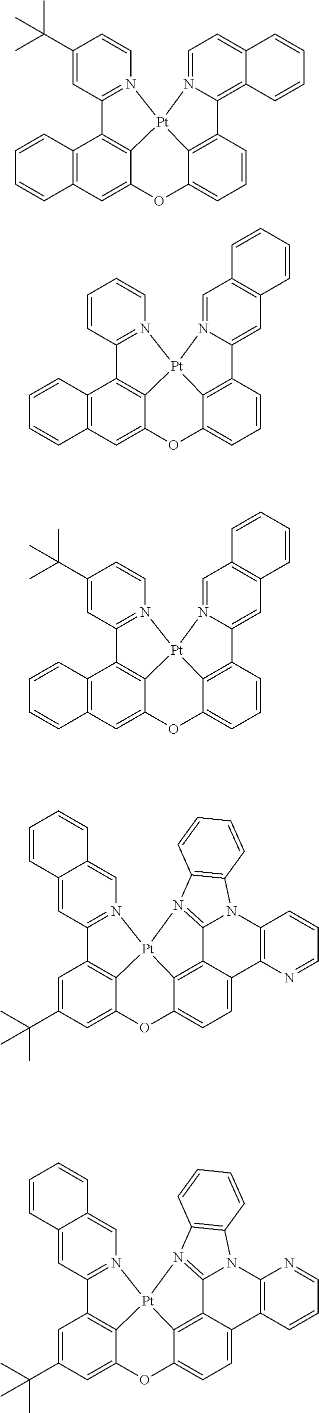

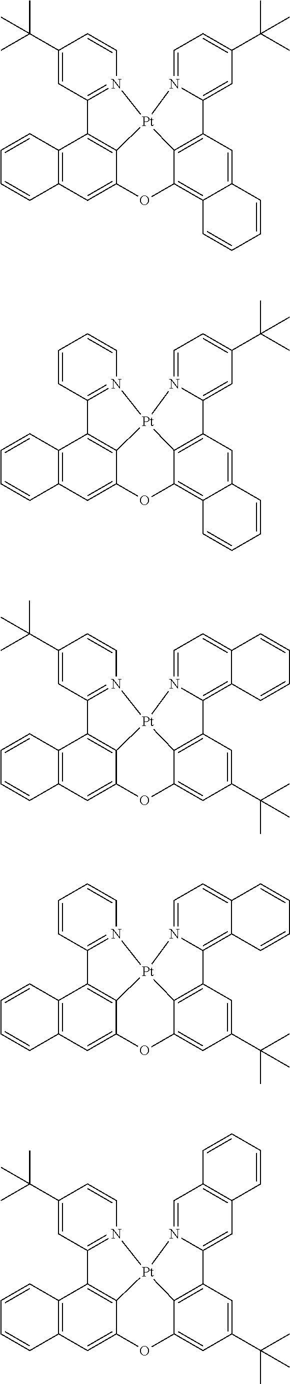

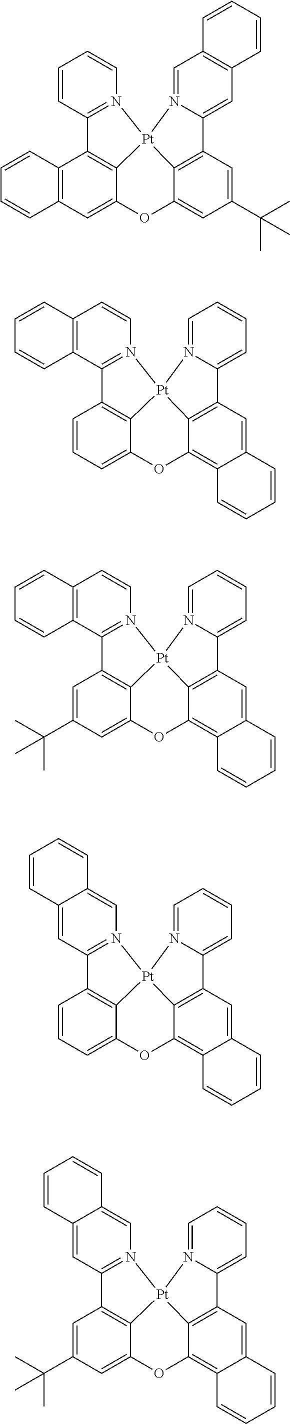

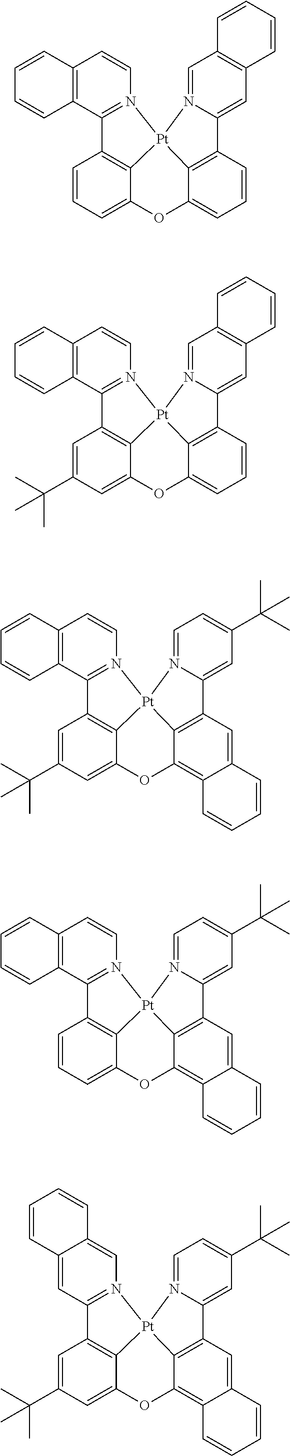

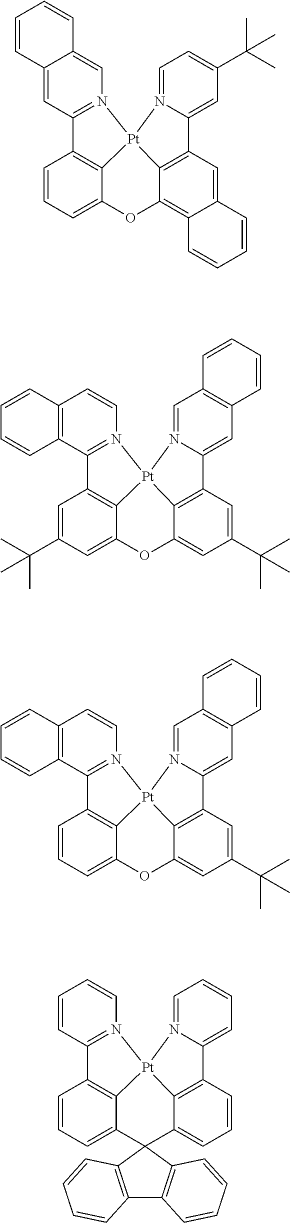

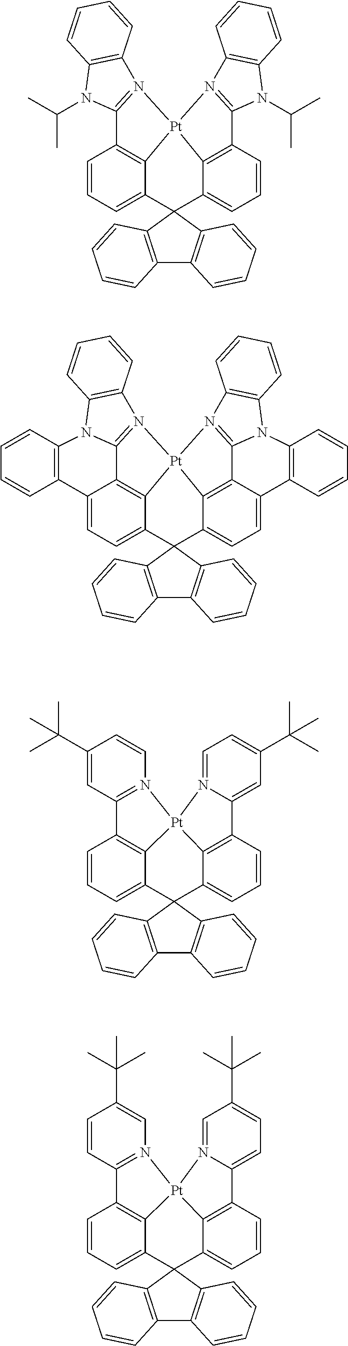

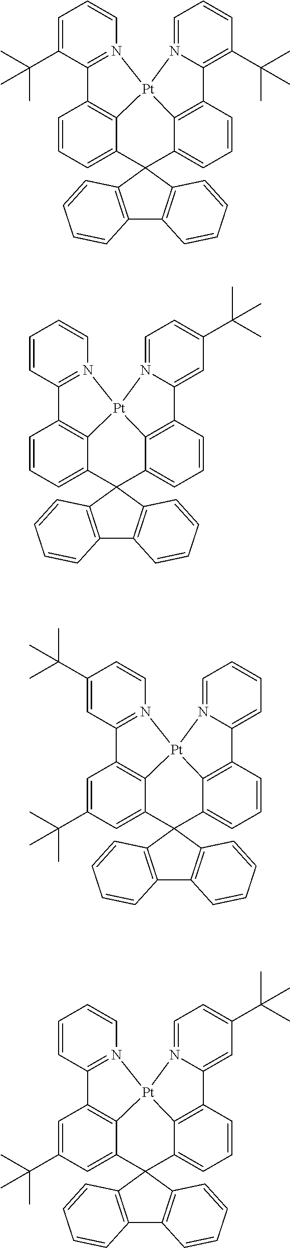

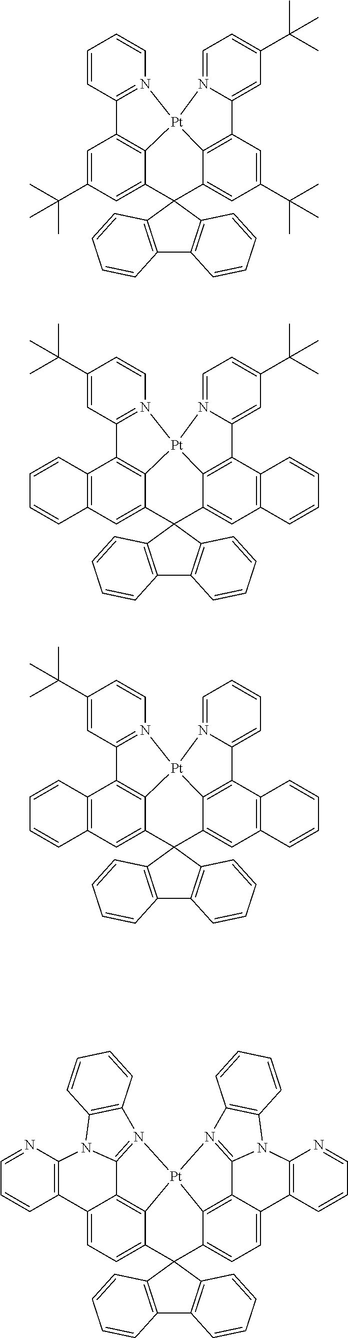

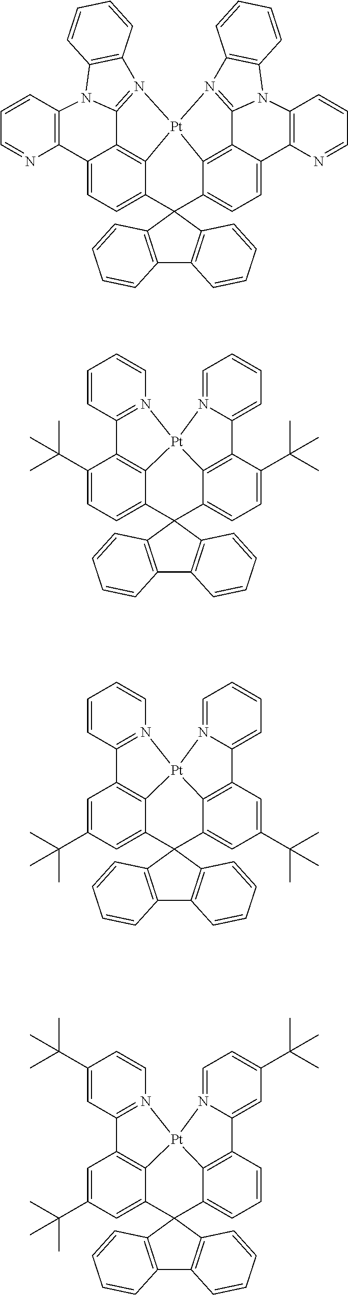

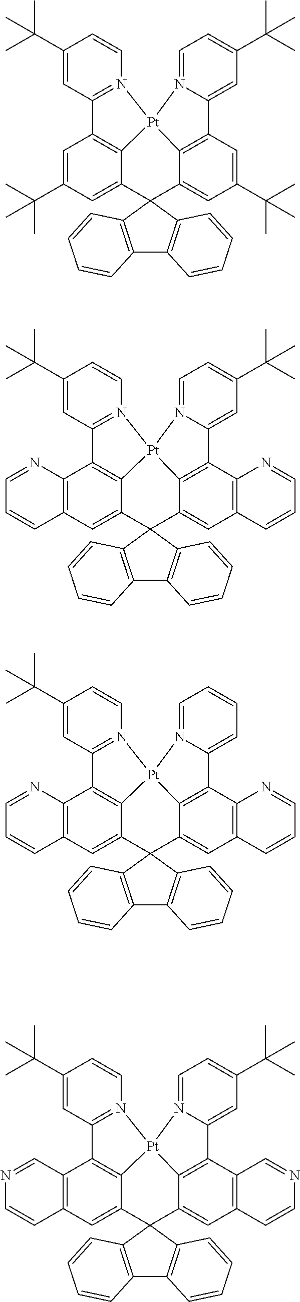

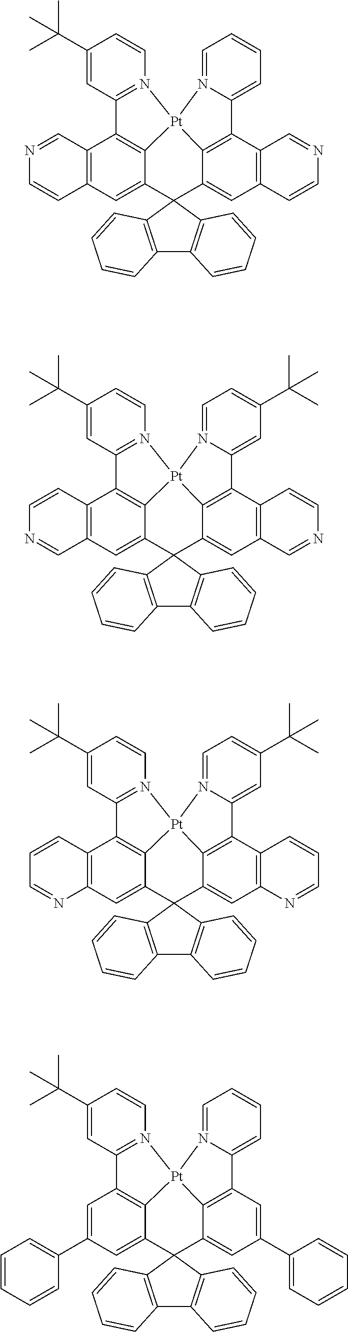

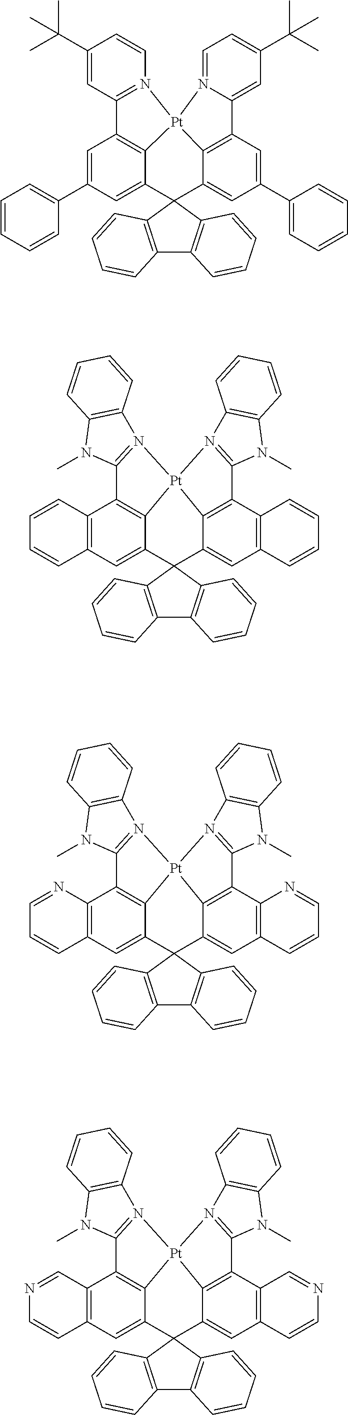

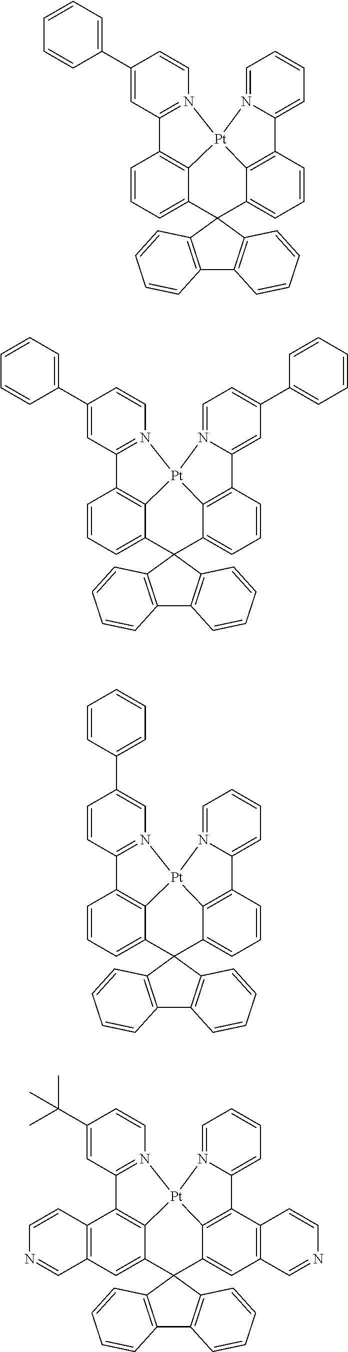

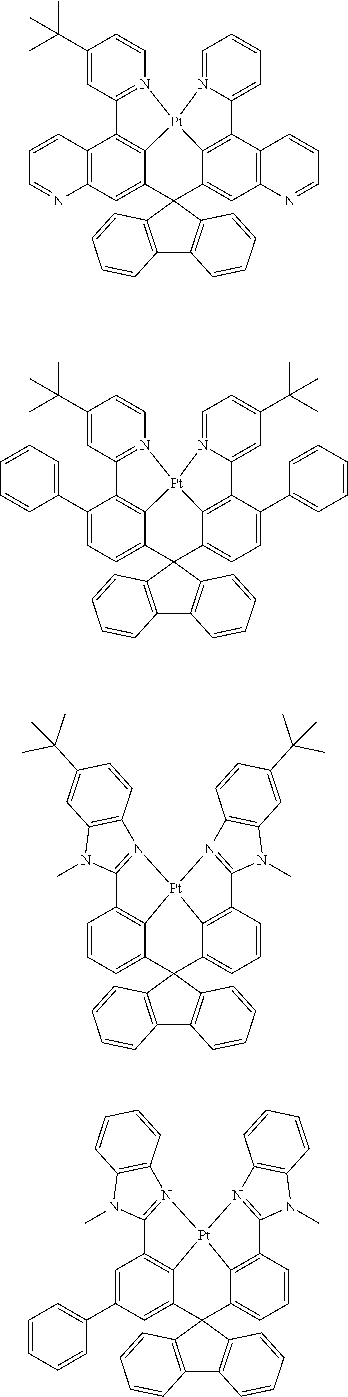

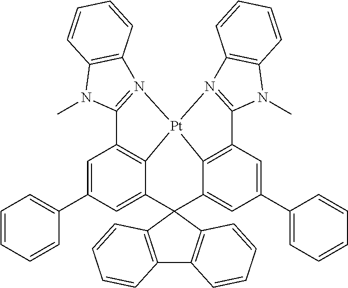

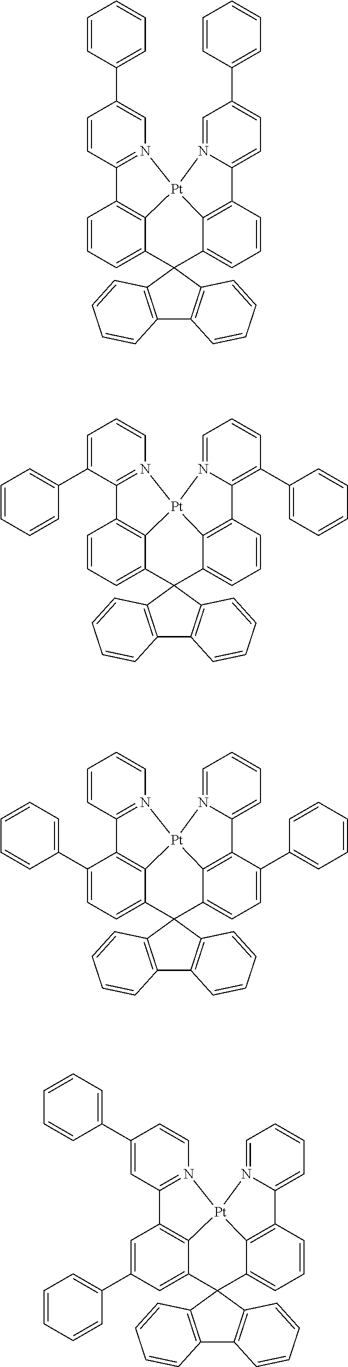

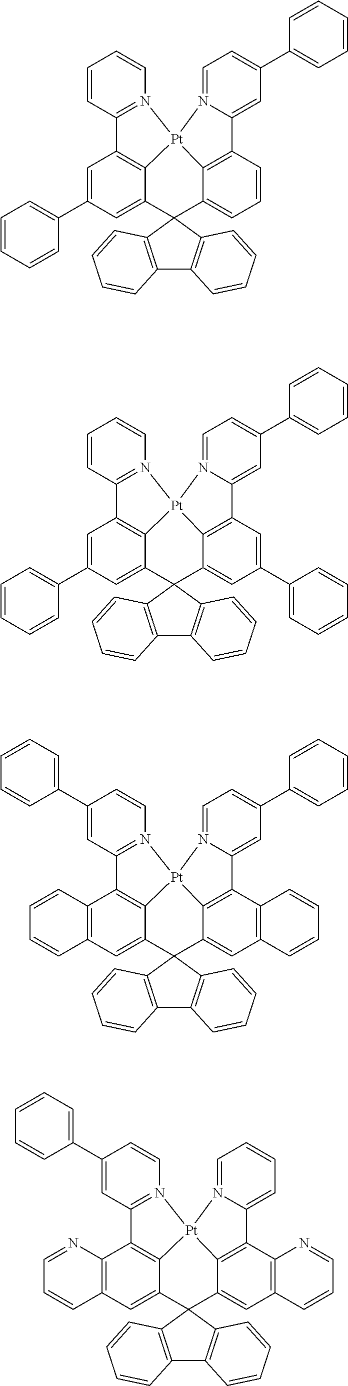

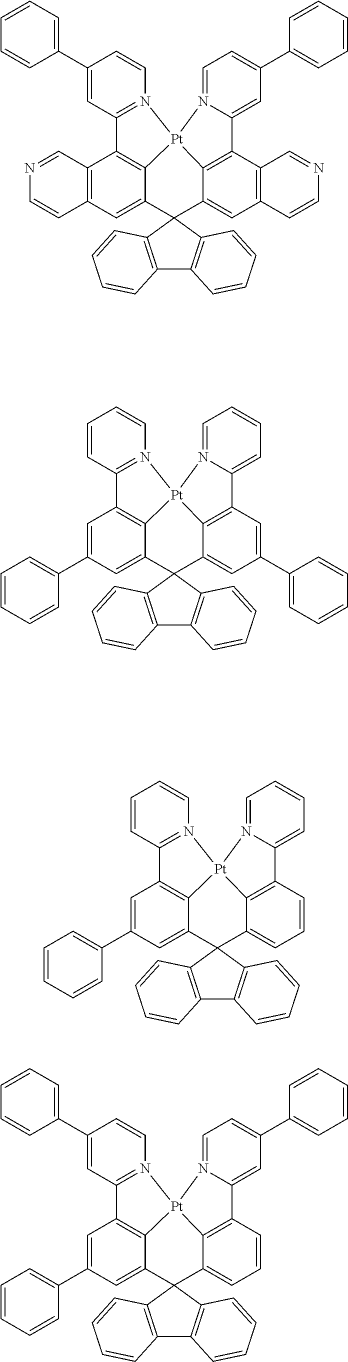

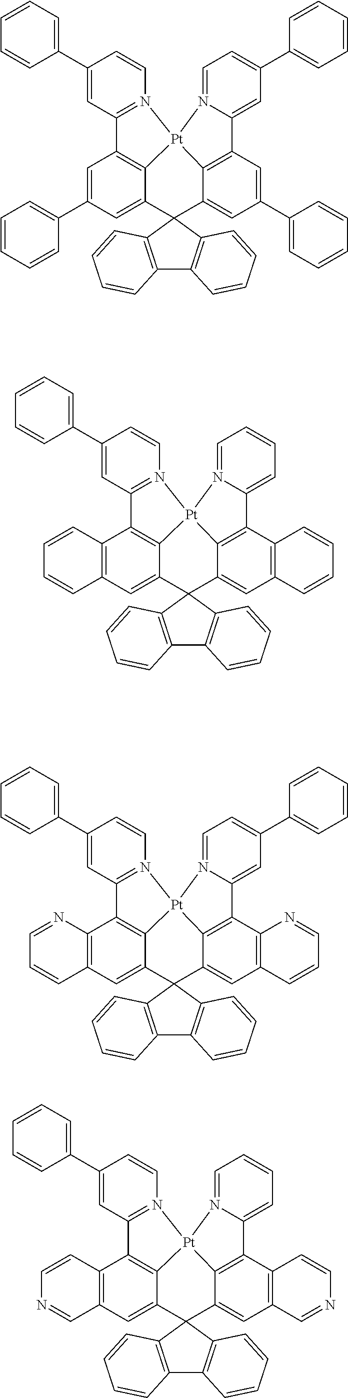

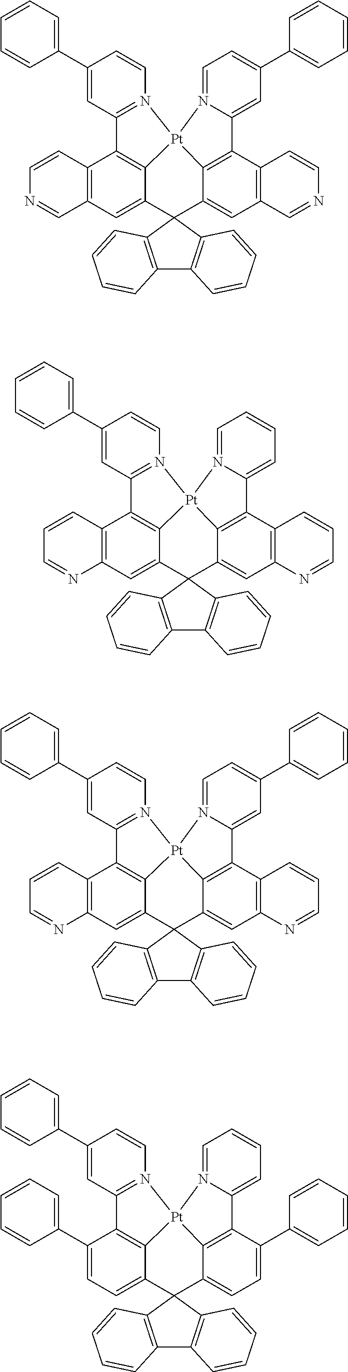

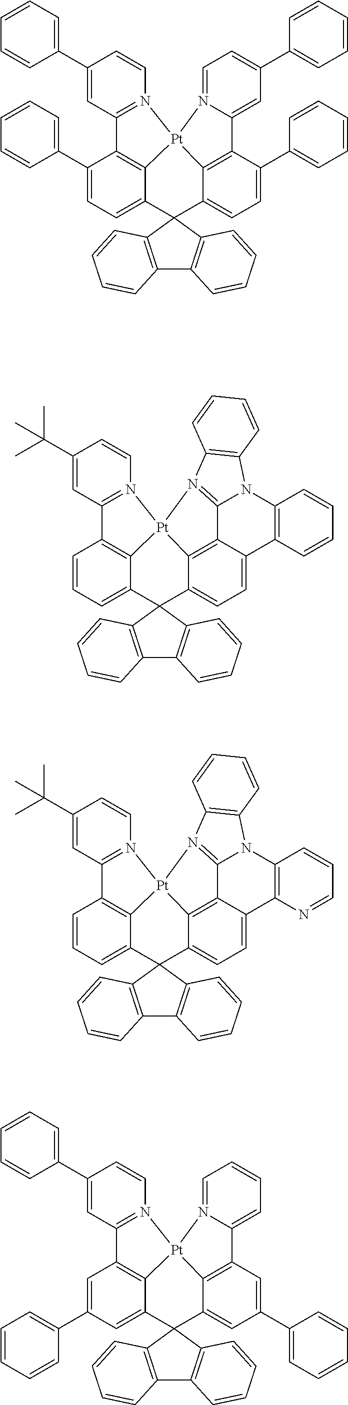

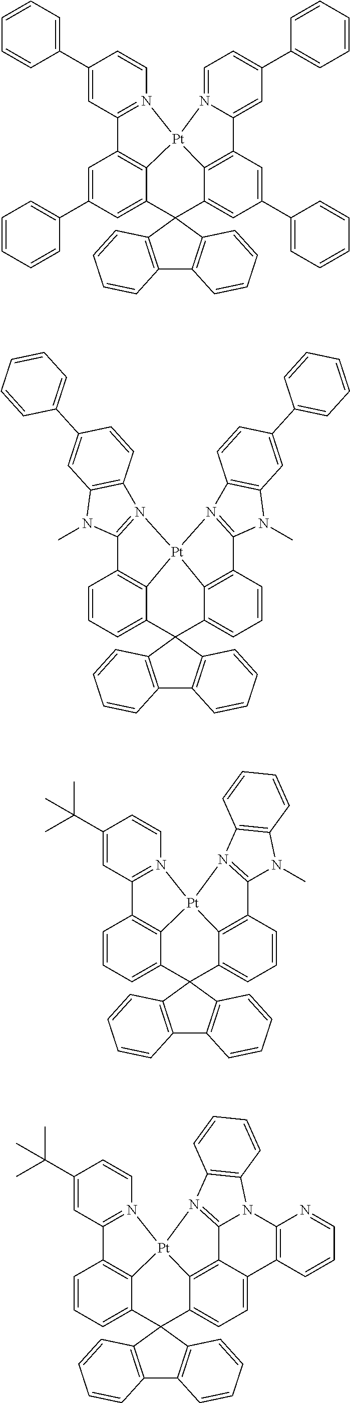

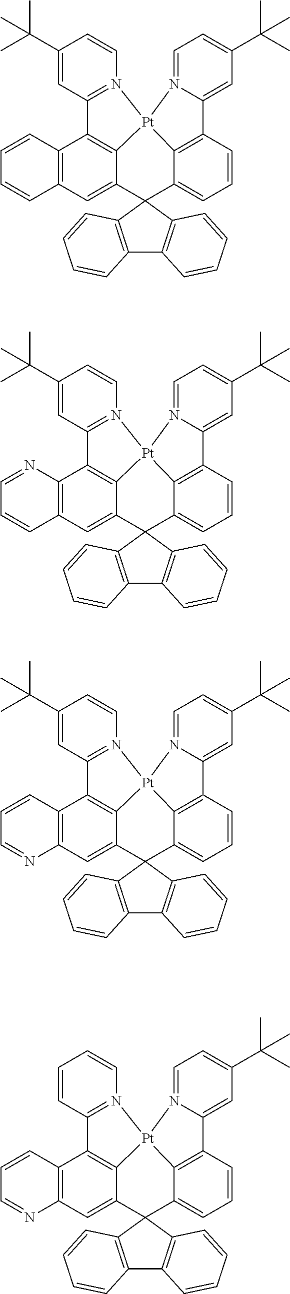

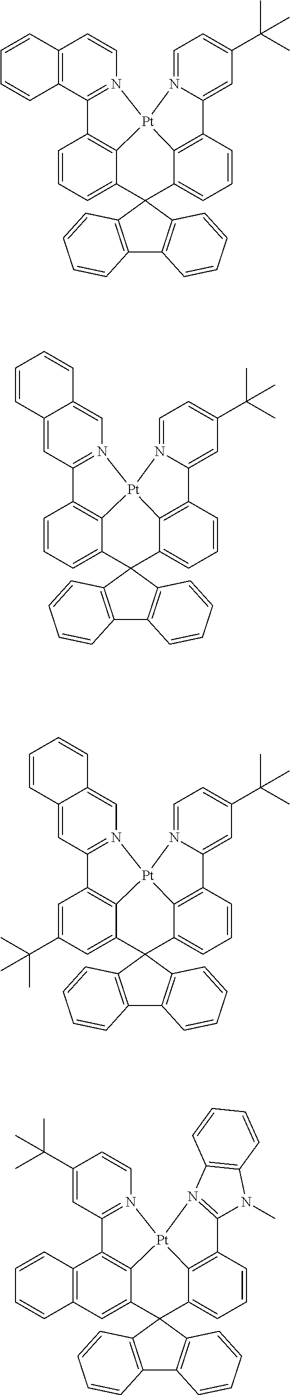

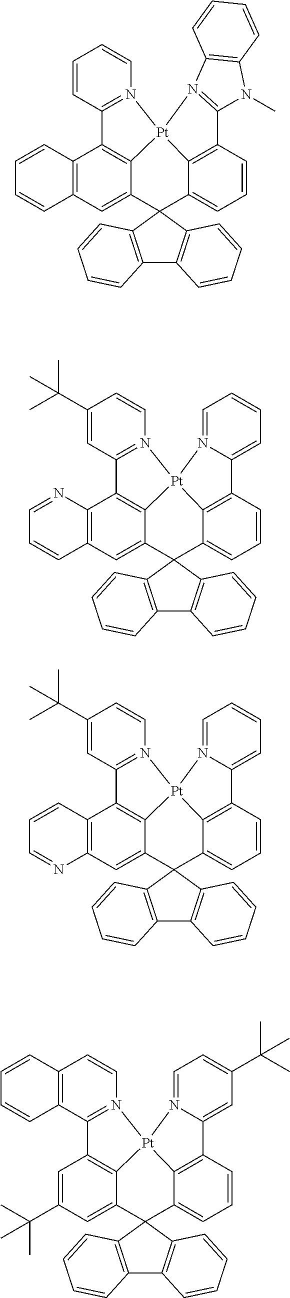

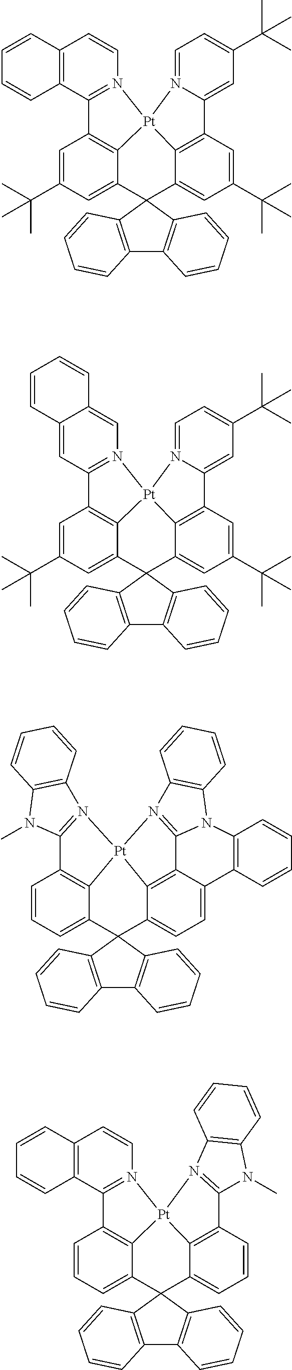

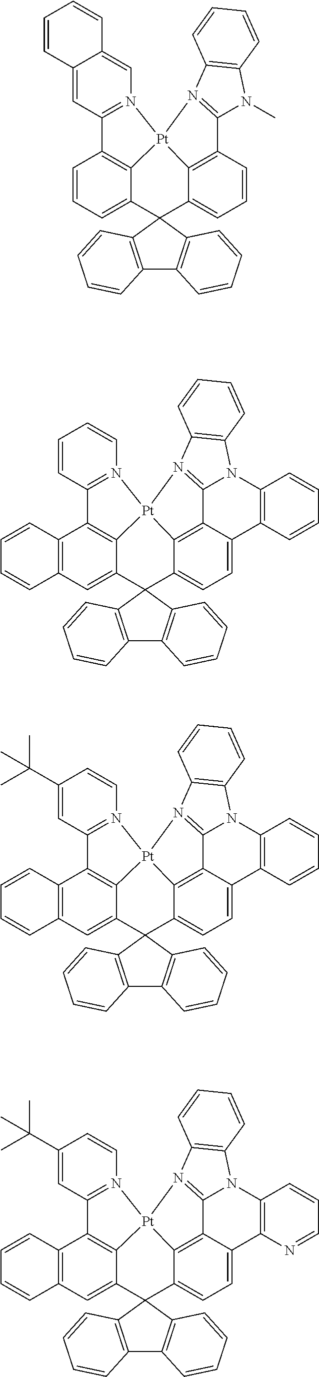

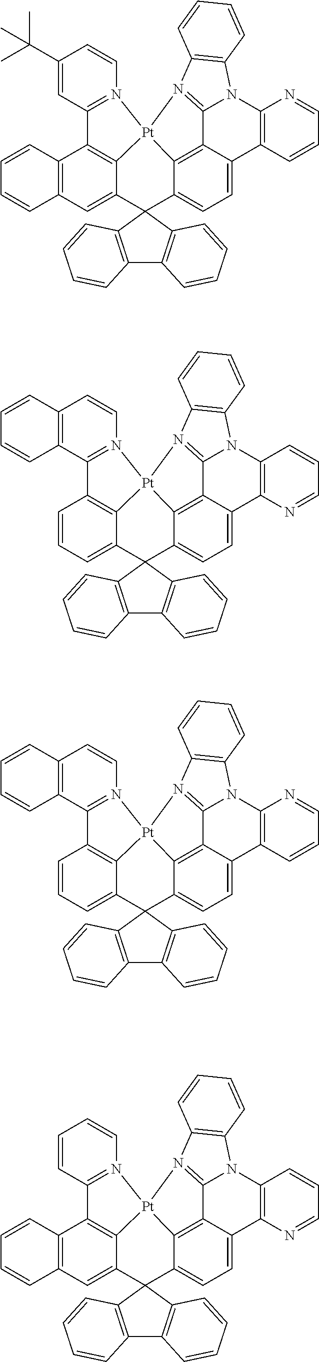

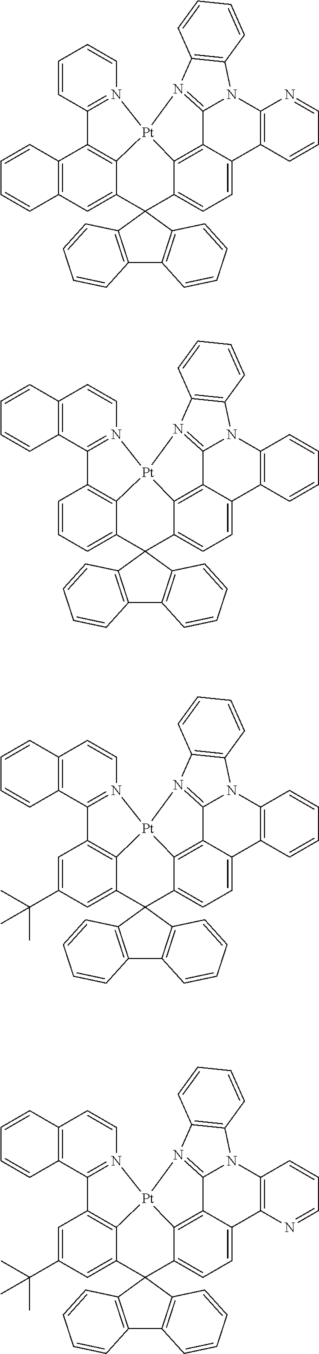

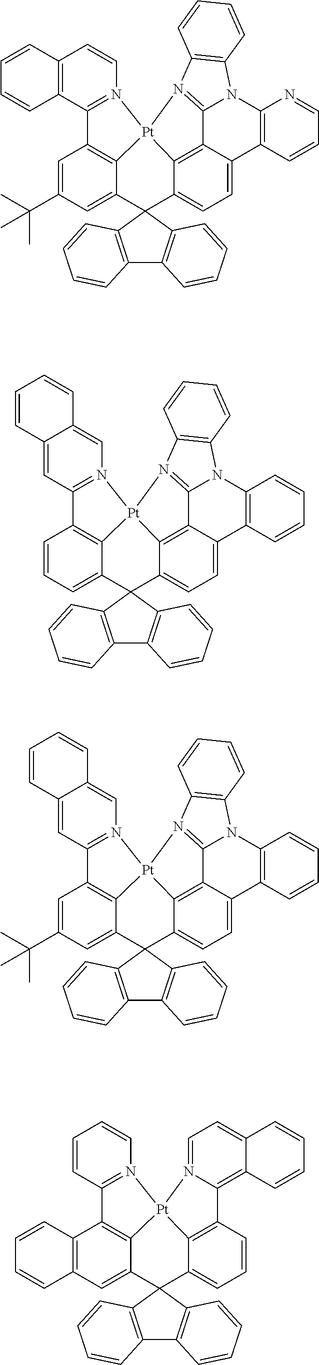

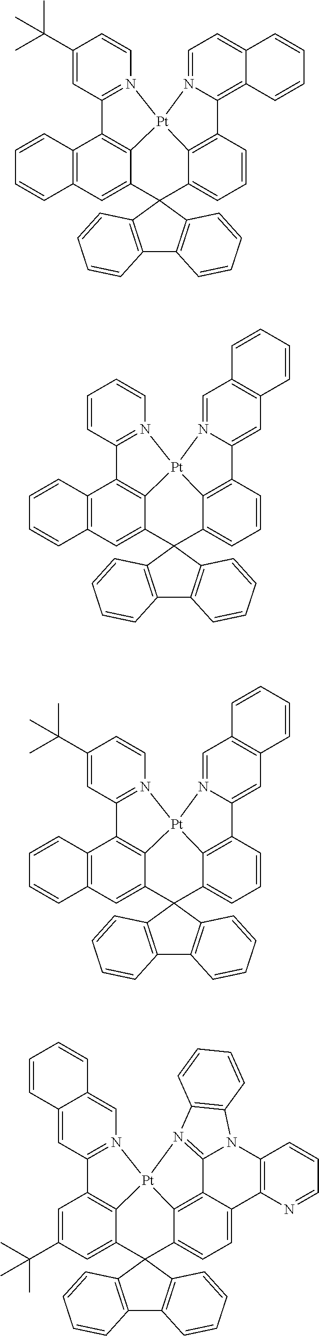

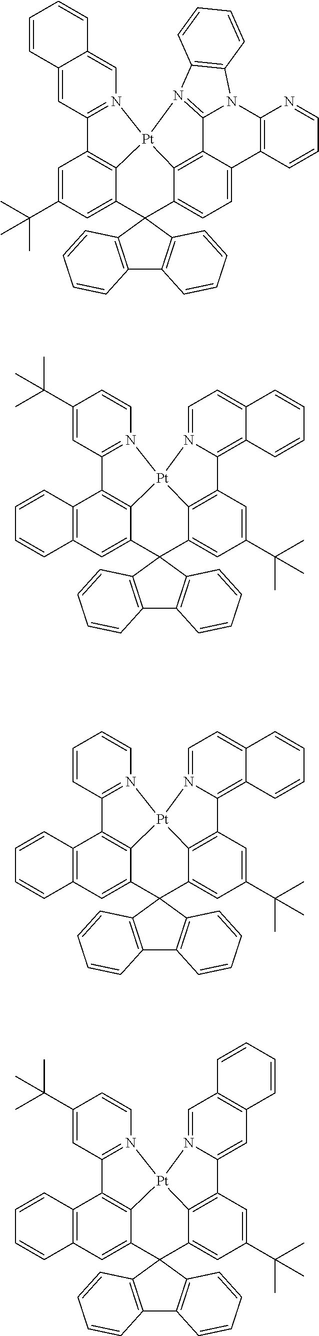

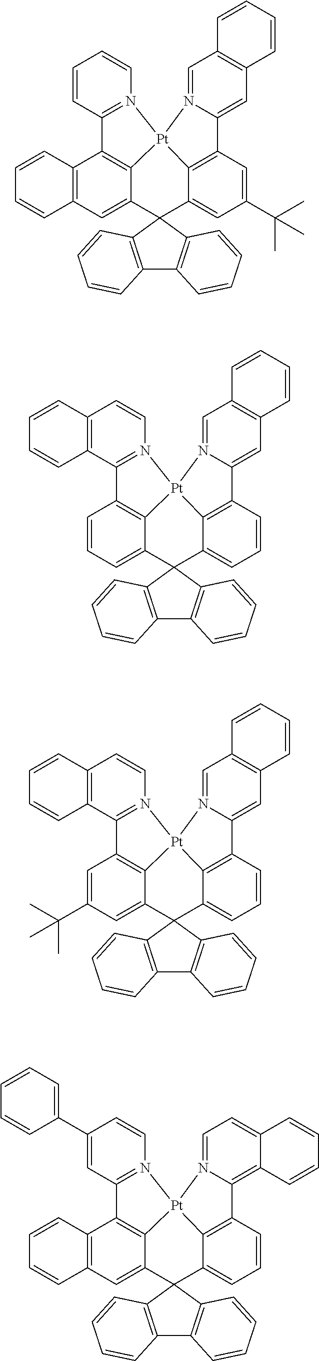

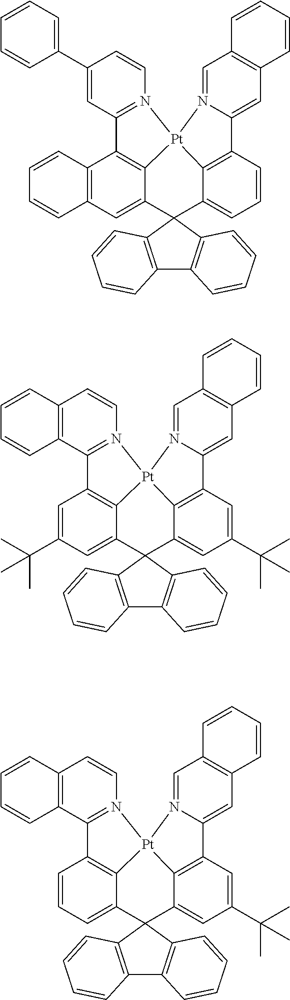

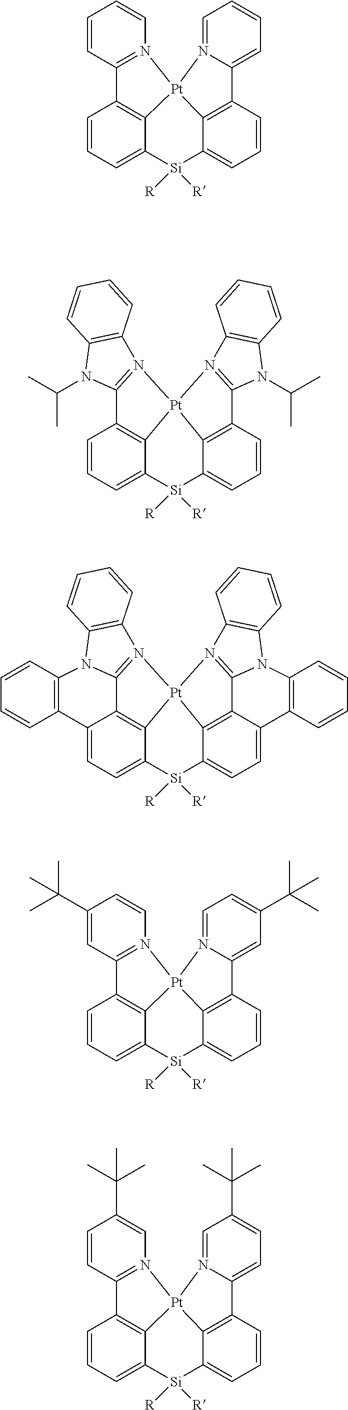

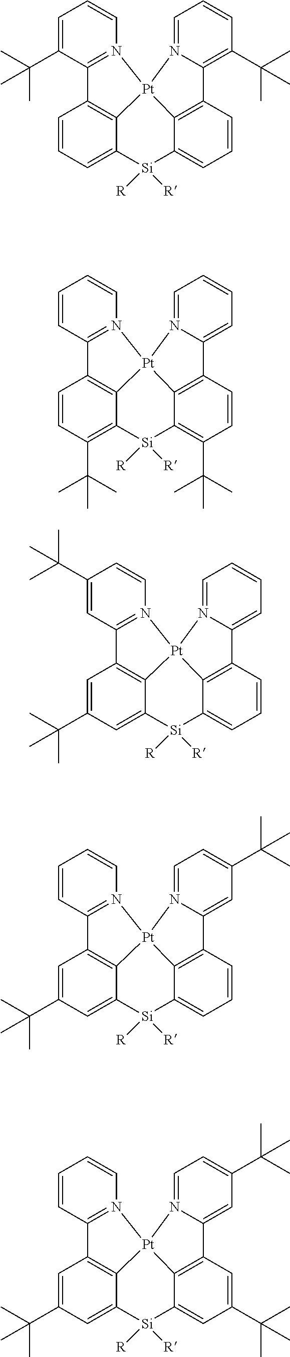

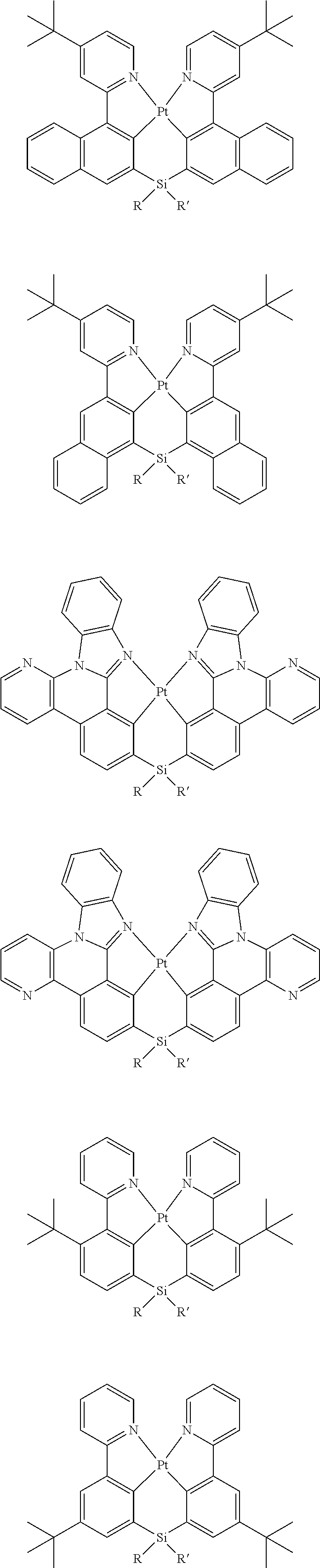

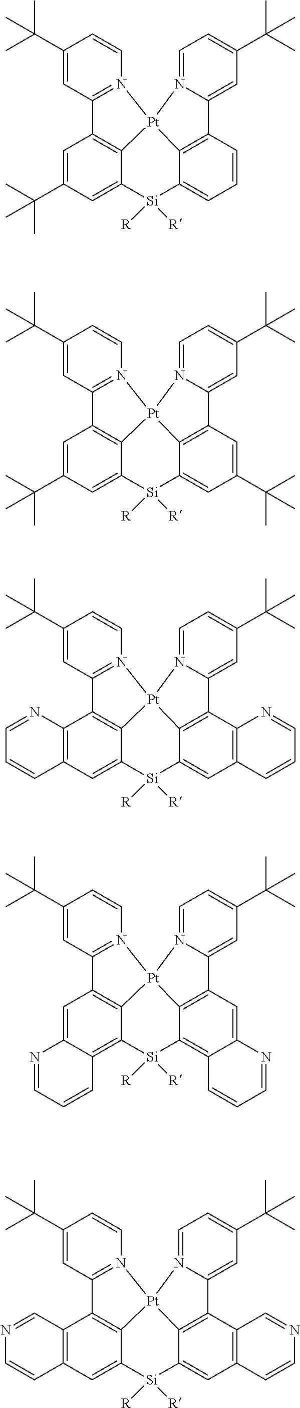

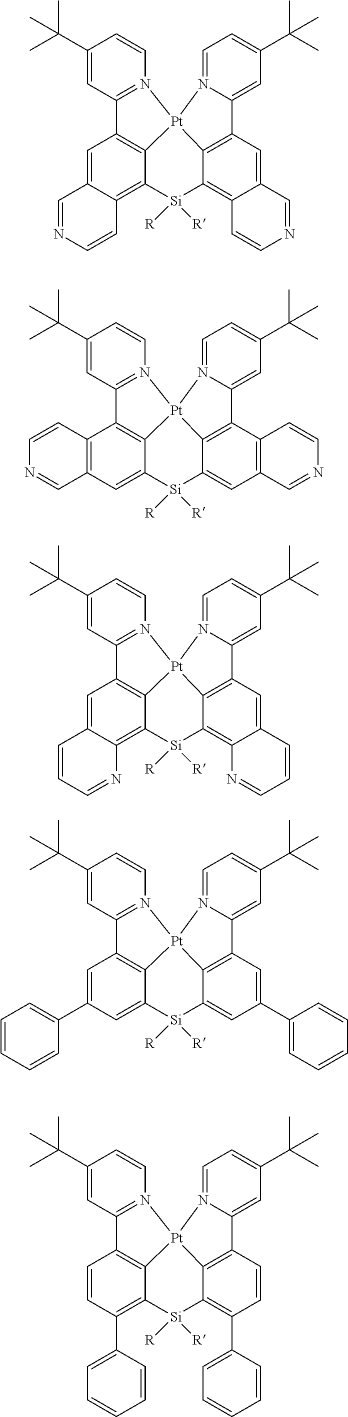

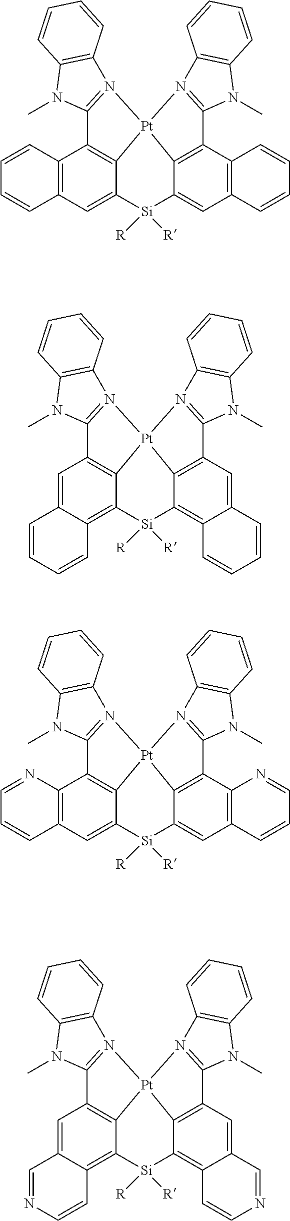

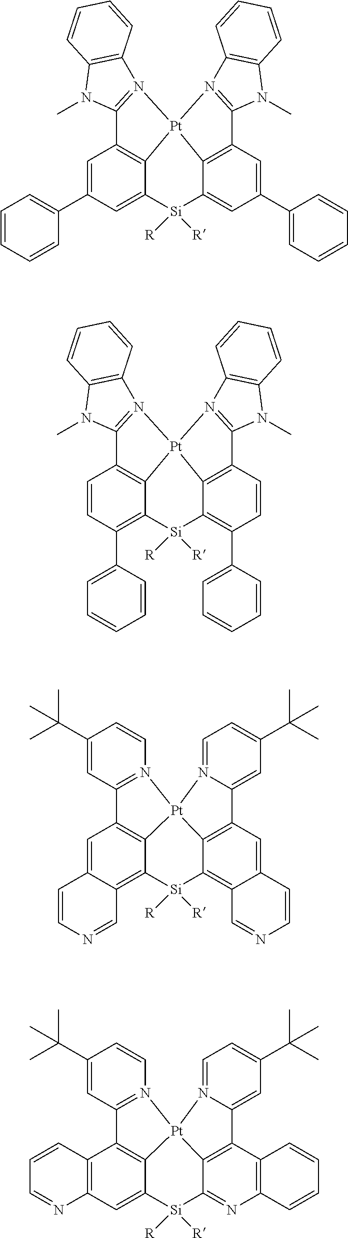

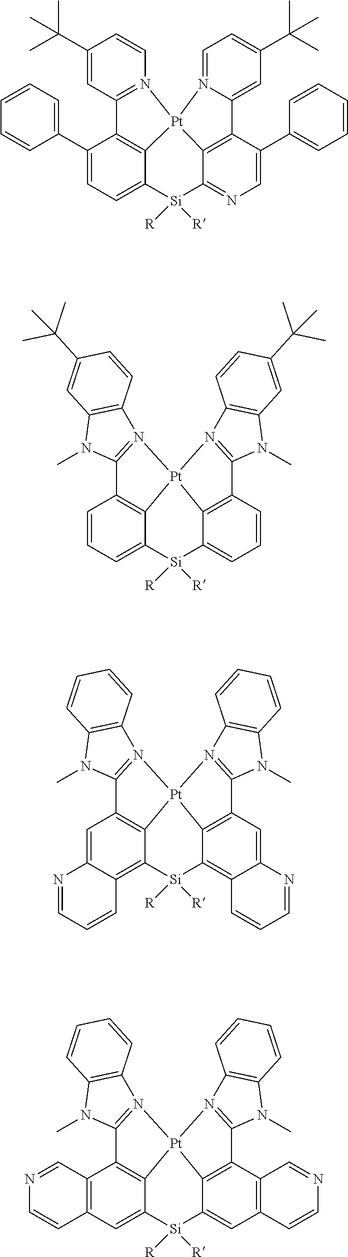

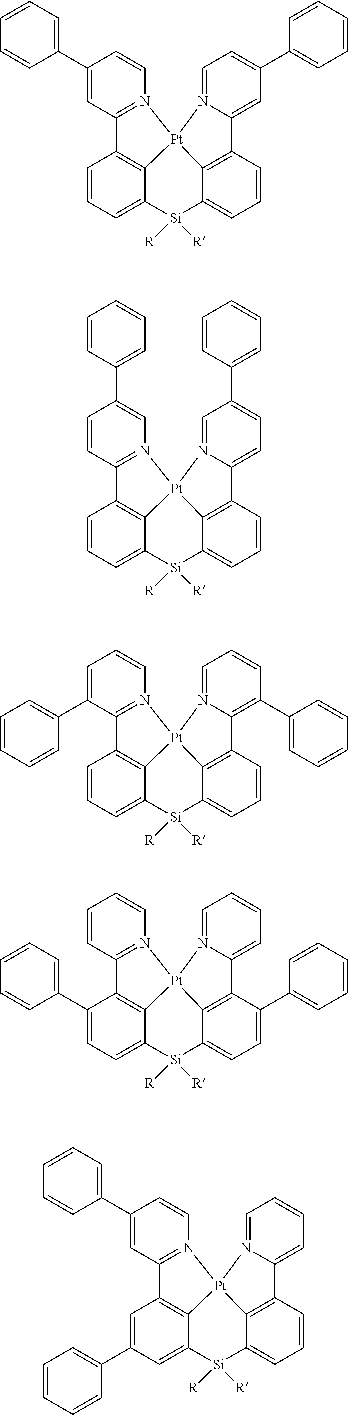

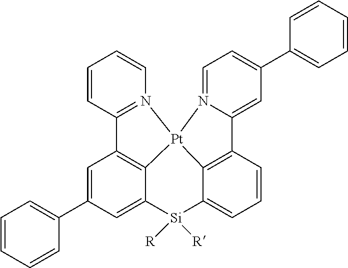

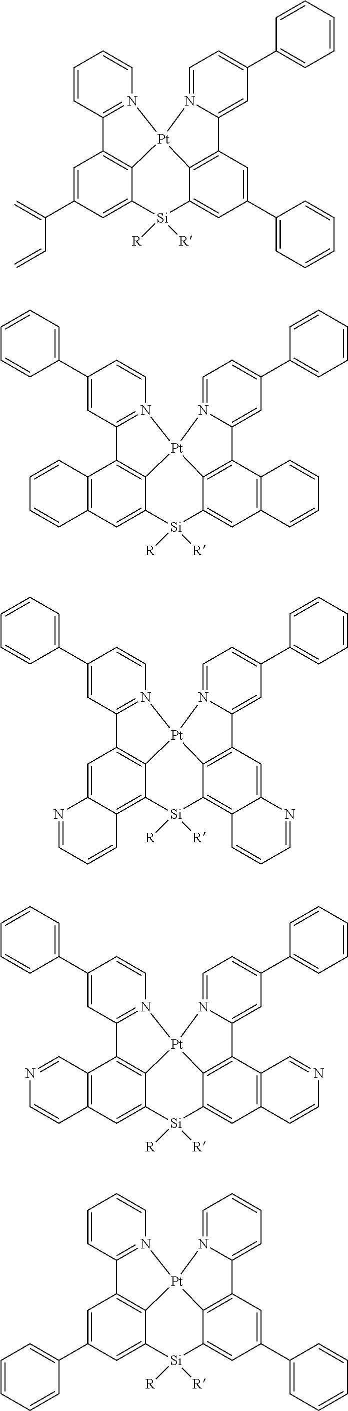

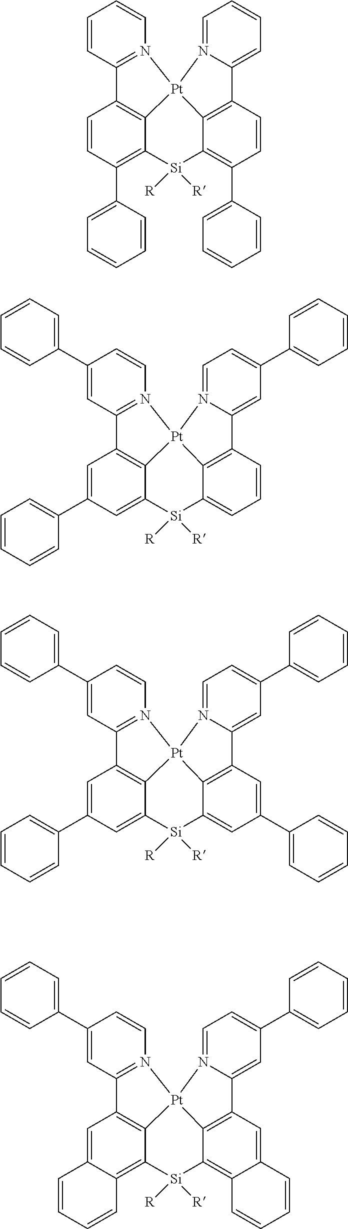

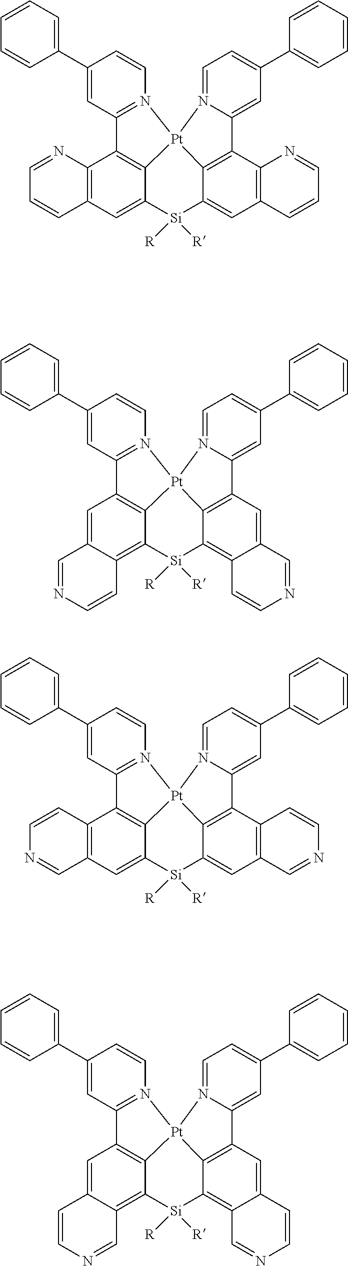

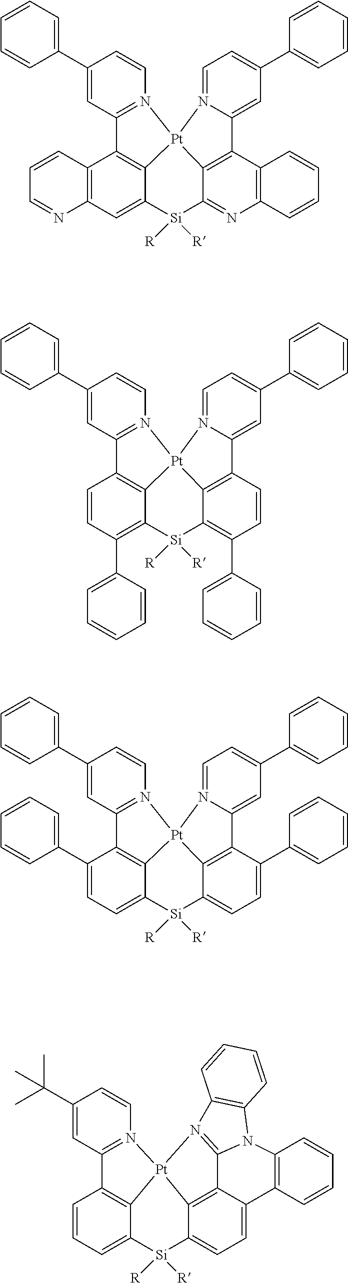

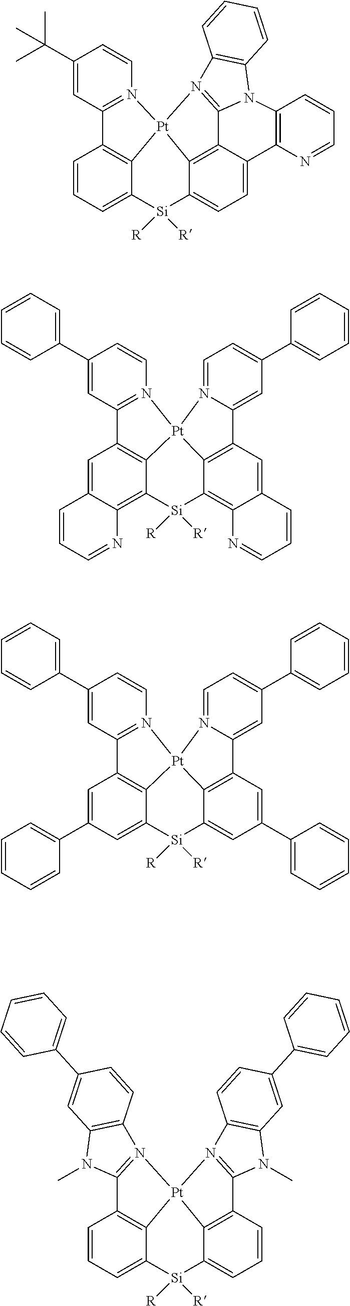

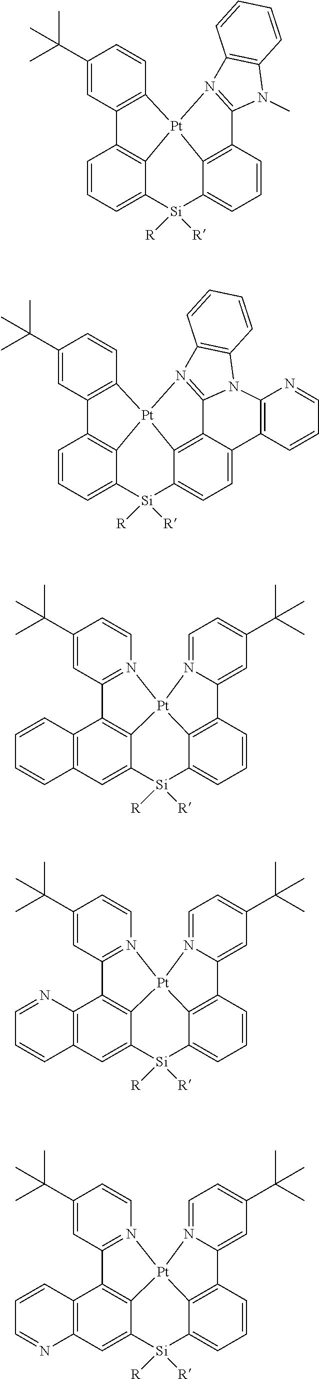

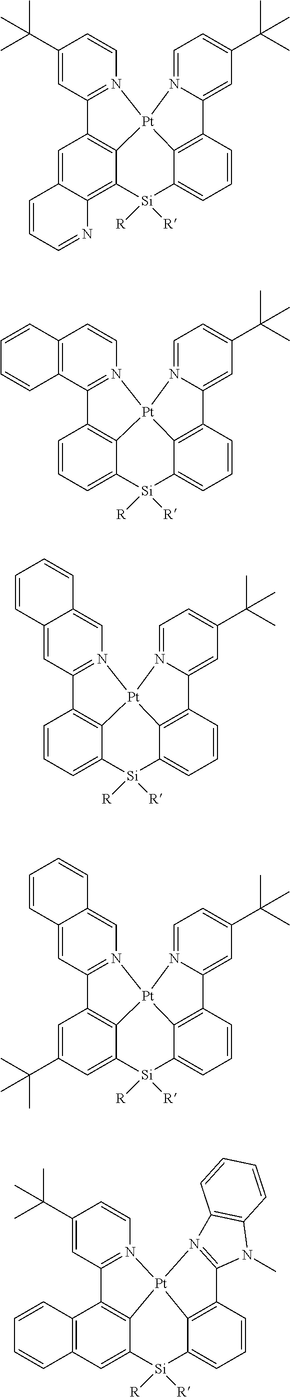

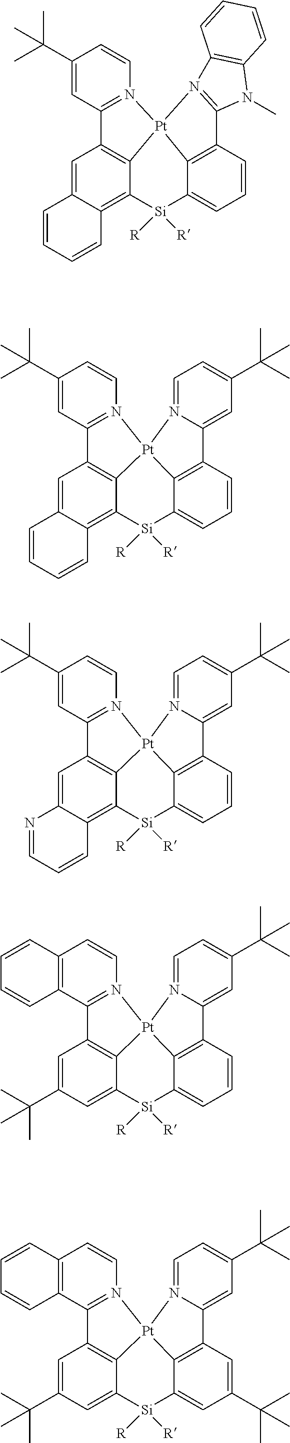

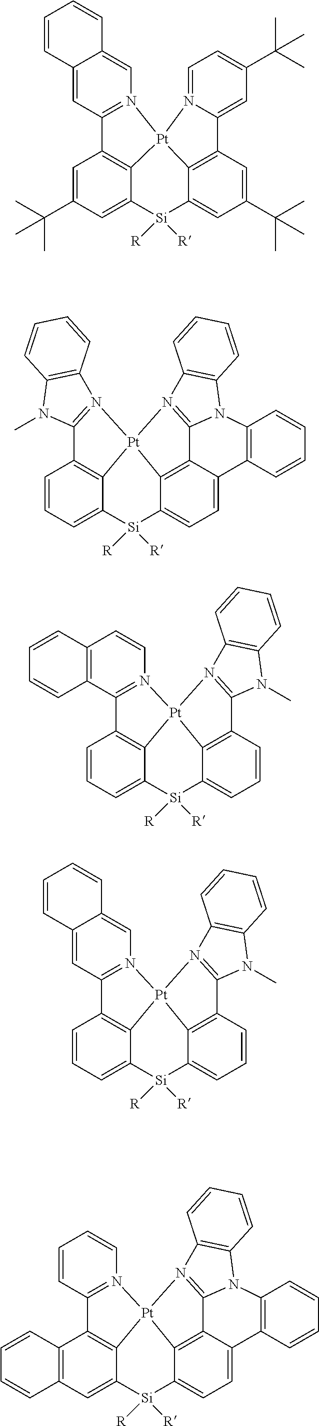

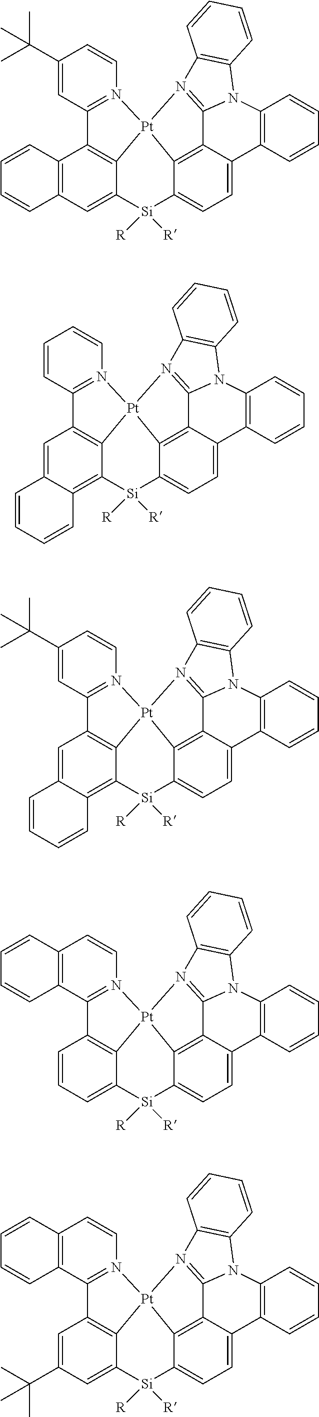

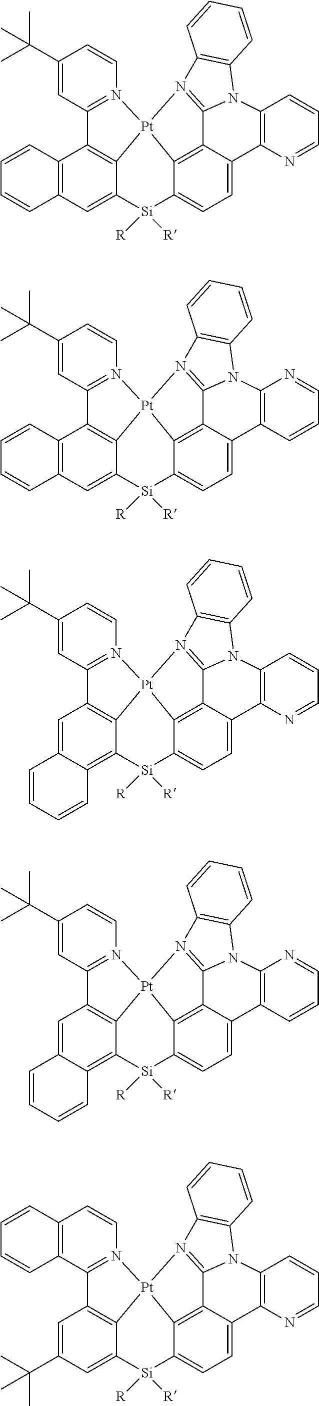

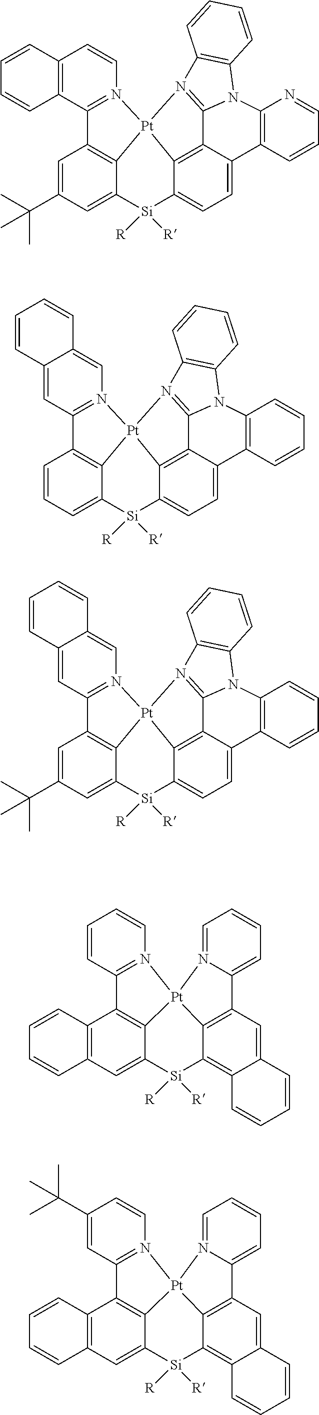

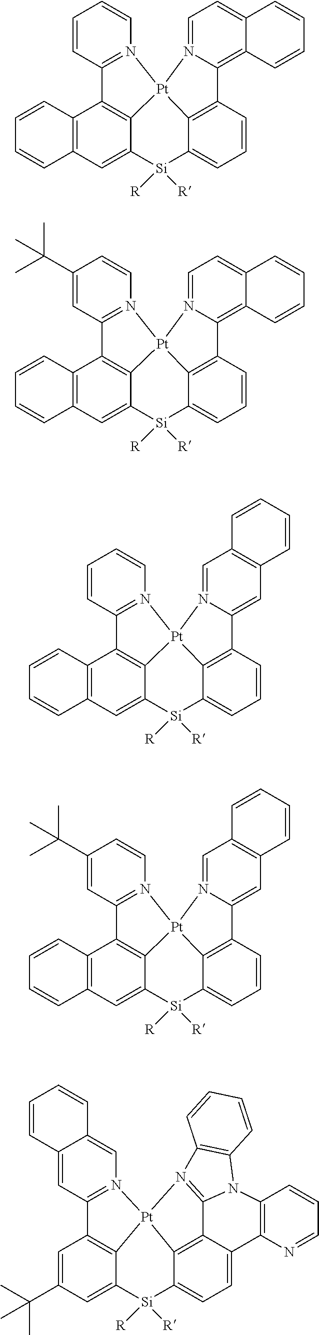

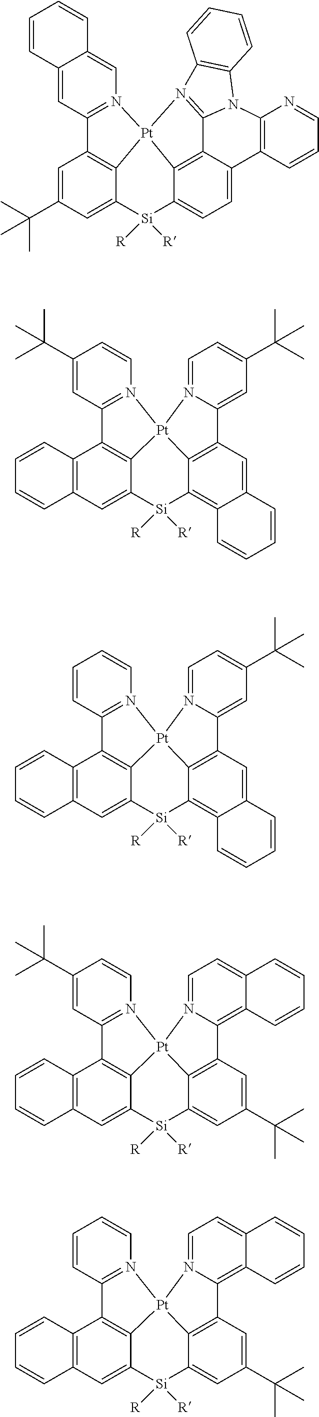

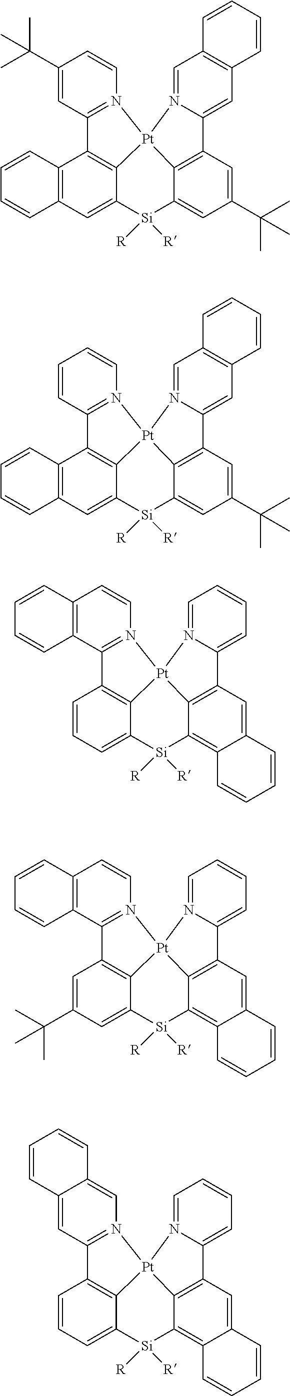

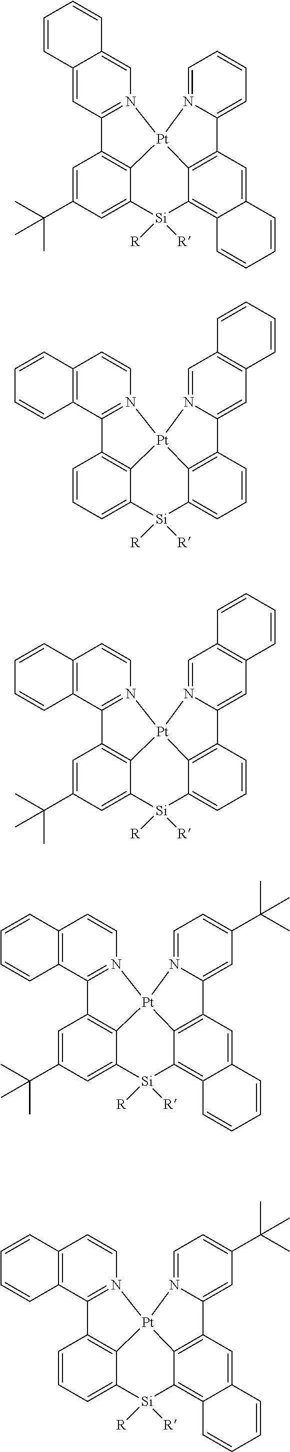

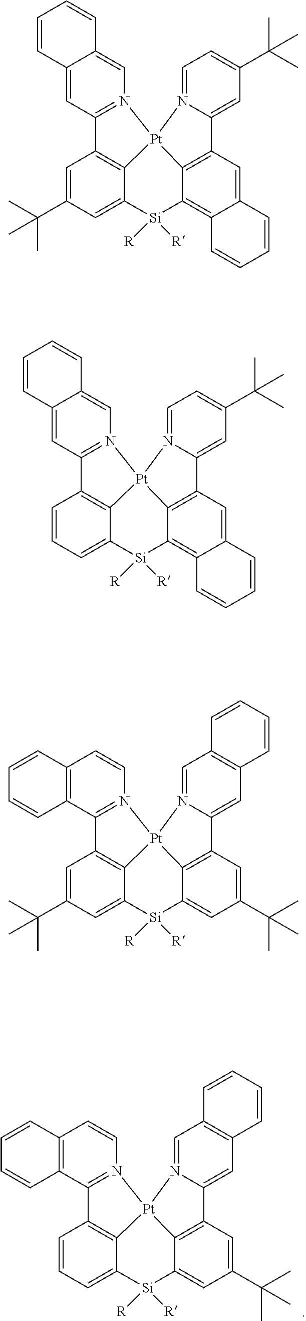

As referred to herein, a linking atom or group connects two atoms such as, for example, an N atom and a C atom. A linking atom or group is in one aspect disclosed as L¹, L², L³, etc. herein. The linking atom can optionally, if valency permits, have other chemical moieties attached. For example, in one aspect, an oxygen would not have any other chemical groups attached as the valency is satisfied once it is bonded to two groups (e.g., N and/or C groups). In another aspect, when carbon is the linking atom, two additional chemical moieties can be attached to the carbon. Suitable chemical moieties include amine, amide, thiol, aryl, heteroaryl, cycloalkyl, and heterocyclyl moieties. The term “cyclic structure” or the like terms used herein refer to any cyclic chemical structure which includes, but is not limited to, aryl, heteroaryl, cycloalkyl, cycloalkenyl, heterocyclyl, carbene, and N-heterocyclic carbene.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹”, “A²”, “A³”, “A⁴” and “A⁵” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” or “halo” as used herein refers to the halogens fluorine, chlorine, bromine, and iodine.

The term “heterocyclyl,” as used herein refers to single and multi-cyclic non-aromatic ring systems and “heteroaryl as used herein refers to single and multi-cyclic aromatic ring systems: in which at least one of the ring members is other than carbon. The terms includes azetidine, dioxane, furan, imidazole, isothiazole, isoxazole, morpholine, oxazole, oxazole, including, 1,2,3-oxadiazole, 1,2,5-oxadiazole and 1,3,4-oxadiazole, piperazine, piperidine, pyrazine, pyrazole, pyridazine, pyridine, pyrimidine, pyrrole, pyrrolidine, tetrahydrofuran, tetrahydropyran, tetrazine, including 1,2,4,5-tetrazine, tetrazole, including 1,2,3,4-tetrazole and 1,2,4,5-tetrazole, thiadiazole, including, 1,2,3-thiadiazole, 1,2,5-thiadiazole, and 1,3,4-thiadiazole, thiazole, thiophene, triazine, including 1,3,5-triazine and 1,2,4-triazine, triazole, including, 1,2,3-triazole, 1,3,4-triazole, and the like.

The term “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Compounds described herein may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain aspects, unless expressly indicated to the contrary, individual substituents can be further optionally substituted (i.e., further substituted or unsubstituted).

In some aspects, a structure of a compound can be represented by a formula:

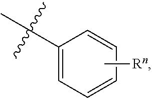

which is understood to be equivalent to a formula:

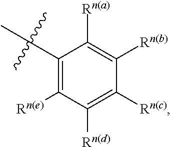

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Several references to R¹, R², R³, R⁴, R⁵, R⁶, etc. are made in chemical structures and moieties disclosed and described herein. Any description of R¹, R², R³, R⁴, R⁵, R⁶, etc. in the specification is applicable to any structure or moiety reciting R¹, R², R³, R⁴, R⁵, R⁶, etc. respectively.

The complexes disclosed herein are suited for use in a wide variety of devices, including, for example, organic light emitting diodes (OLEDs) for full color displays and lighting applications.

Also disclosed herein are compositions including one or more complexes disclosed herein. The present disclosure provides light emitting devices that include one or more compositions described herein. The present disclosure also provides a photovoltaic device comprising one or more complexes or compositions described herein. Further, the present disclosure also provides a luminescent display device comprising one or more complexes described herein.

Complexes described herein can be used in a light emitting device such as an OLED. FIG. 2 depicts a cross-sectional view of an OLED 100. OLED 100 includes substrate 102, anode 104, hole-transporting material(s) (HTL) 106, light processing material 108, electron-transporting material(s) (ETL) 110, and a metal cathode layer 112. Anode 104 is typically a transparent material, such as indium tin oxide. Light processing material 108 may be an emissive material (EML) including an emitter and a host.

In various aspects, any of the one or more layers depicted in FIG. 2 may include indium tin oxide (ITO), poly(3,4-ethylenedioxythiophene) (PEDOT), polystyrene sulfonate (PSS), N,N′-di-1-naphthyl-N,N-diphenyl-1,1′-biphenyl-4,4′diamine (NPD), 1,1-bis((di-4-tolylamino)phenyl)cyclohexane (TAPC), 2,6-Bis(N-carbazolyl)pyridine (mCpy), 2,8-bis(diphenylphosphoryl)dibenzothiophene (PO15), LiF, Al, or a combination thereof.

Light processing material 108 may include one or more complexes of the present disclosure optionally together with a host material. The host material can be any suitable host material known in the art. The emission color of an OLED is determined by the emission energy (optical energy gap) of the light processing material 108, which can be tuned by tuning the electronic structure of the emitting complexes, the host material, or both. Both the hole-transporting material in the HTL layer 106 and the electron-transporting material(s) in the ETL layer 110 may include any suitable hole-transporter known in the art.

### Examples

The following examples are put forth so as to provide those of ordinary skill in the art with a complete disclosure and description of how the complexes, compositions, articles, devices and/or methods claimed herein are made and evaluated, and are intended to be purely exemplary and are not intended to be limiting in scope. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Various methods for the preparation method of the complexes described herein are recited in the examples. These methods are provided to illustrate various methods of preparation, but are not intended to limit any of the methods recited herein. Accordingly, one of skill in the art in possession of this disclosure could readily modify a recited method or utilize a different method to prepare one or more of the complexes described herein. The following aspects are only exemplary and are not intended to be limiting in scope. Temperatures, catalysts, concentrations, reactant compositions, and other process conditions can vary, and one of skill in the art, in possession of this disclosure, could readily select appropriate reactants and conditions for a desired complex.

General Procedure for Pt Complexes with General Formulas I-VIII

To a solution of corresponding ligand (1 eq) in HOAc (0.02 M) were added K₂PtCl₄ (1.1 eq) and n-Bu₄NBr (0.1 eq). The mixture was heated to reflux for 3 days. The reaction mixture was cooled to room temperature and filtered through a short pad of silica gel. The filtrate was concentrated under reduced pressure. Purification by column chromatography (hexanes:DCM) gave the corresponding complexes.

### Synthetic Route for PtNON-S56

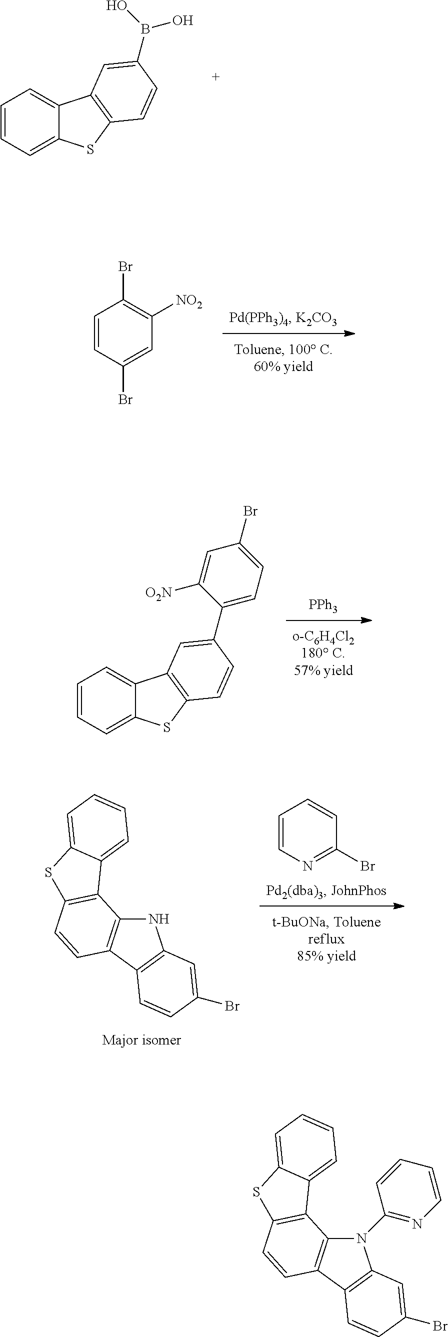

### Suzuki Coupling of Aryl Boronic Acid and Aryl Bromide

To an oven-dried flask were added dibenzo[b,d]thiophen-2-ylboronic acid (1.0 eq), 1,4-dibromo-2-nitrobenzene (1.1 eq), and Pd(PPh₃)₄ (0.08 eq). The flask was then evacuated and backfilled with nitrogen 3 times. Aqueous K₂CO₃ (2 M, 3 eq) and toluene (0.2 M) were then added. The mixture was heated to 100° C. for about 4 hours. The mixture was cooled to room temperature and the product was isolated by column chromatography (Hexane:EtOAc=20:1 to 10:1) in 60% yield.

### PPh₃-Mediated Reductive Cyclization

To an oven-dried flask were added 2-(4-bromo-2-nitrophenyl)dibenzo[b,d]-thiophene (1.0 eq) and PPh₃ (2.5 eq). The flask was then evacuated and backfilled with nitrogen 3 times, following which 1,2-dichlorobenzene (0.25 M) was added by syringe. The mixture was heated with 180° C. oil bath for 12 hours. The mixture was cooled to room temperature and the product was isolated by column chromatography (Hexane:EtOAc=15:1 to 8:1) in 57% yield.

### Pd-Catalyzed Coupling of Carbazole and 2-Bromopyridine

To an oven-dried flask were added 10-bromo-12H-benzo[4,5]thieno[3,2-a]carbazole (1 eq), 2-bromopyridine (2 eq), Pd₂(dba)₃ (0.05 eq), JohnPhos (0.1 eq) and t-BuONa (1.5 eq). The flask was then evacuated and backfilled with nitrogen 3 times, following which toluene (0.1 M) was added by syringe. The mixture was heated to reflux for 12 hours. The mixture was cooled to room temperature and the product was isolated by column chromatography (Hexane:EtOAc=15:1 to 8:1) in 85% yield.

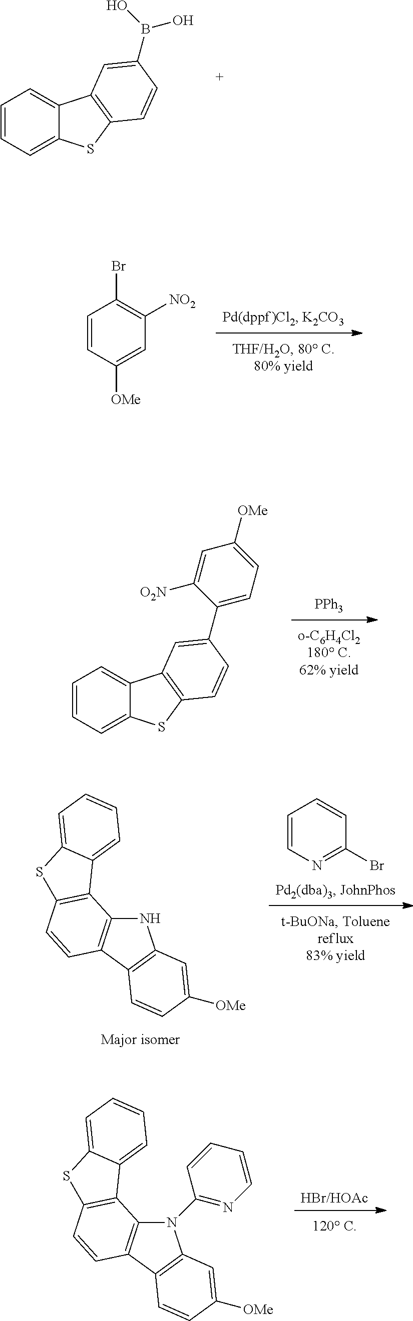

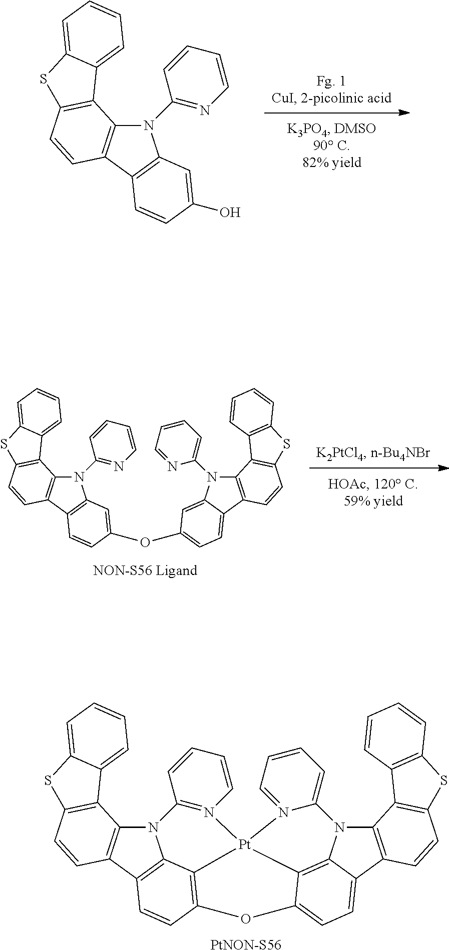

### Suzuki Coupling of Aryl Boronic Acid and Aryl Bromide

To an oven-dried flask were added dibenzo[b,d]thiophen-2-ylboronic acid (1.1 eq), 1-bromo-4-methoxy-2-nitrobenzene (1 eq), and Pd(PPh₃)₄ (0.08 eq). The flask was then evacuated and backfilled with nitrogen 3 times. Aqueous K₂CO₃ (2 M, 3 eq) and THF (0.2 M) were then added. The mixture was heated with 80° C. oil bath for about 4 hours. The mixture was cooled to room temperature and the product was isolated by column chromatography (Hexane:EtOAc=15:1 to 8:1) in 80% yield.

### PPh₃-Mediated Reductive Cyclization

To an oven-dried flask were added 2-(4-methoxy-2-nitrophenyl)dibenzo[b,d]-thiophene (1.0 eq) and PPh₃ (2.5 eq). The flask was then evacuated and backfilled with nitrogen 3 times, following which 1,2-dichlorobenzene (0.25 M) was added by syringe. The mixture was heated with 180° C. oil bath for 12 hours. The mixture was cooled to room temperature and the product was isolated by column chromatography (Hexane:EtOAc=10:1 to 6:1) in 62% yield.

### Pd-Catalyzed Coupling of Carbazole and 2-Bromopyridine

To an oven-dried flask were added 10-methoxy-12H-benzo[4,5]thieno[3,2-a]carbazole (1 eq), 2-bromopyridine (2 eq), Pd₂(dba)₃ (0.05 eq), JohnPhos (0.1 eq) and t-BuONa (1.5 eq). The flask was then evacuated and backfilled with nitrogen 3 times, following which toluene (0.1 M) was added by syringe. The mixture was heated to reflux for 12 hours. The mixture was cooled to room temperature and the product was isolated by column chromatography (Hexane:EtOAc=10:1 to 6:1) in 83% yield.

### Demethylation of 10-methoxy-12-(pyridin-2-yl)-12H-benzo[4,5]thieno[3,2-a]carbazole

To a solution of 10-methoxy-12-(pyridin-2-yl)-12H-benzo[4,5]thieno[3,2-a]carbazole in acetic acid (0.1 M) was added aqueous HBr (48 wt %, 10 eq). The mixture was heated with 120° C. oil bath for 12 hours. The mixture was cooled to room temperature and water (equal volume to acetic acid) was then added. The mixture was neutralized with solid K₂CO₃ to pH 5-6. The precipitate was collected by filtration, rinsed with water 3 times, and dried under reduced pressure. The yield was quantitative.

### CuI-Catalyzed Synthesis of NON-S56 Ligand

To a solution of 12-(pyridin-2-yl)-12H-benzo[4,5]thieno[3,2-a]carbazol-10-ol (1 eq) in DMSO (0.1 M) were added FIG. 1 (1.2 eq), CuI (0.1 eq), 2-picolinic acid (0.2 eq) and K₃PO₄ (2 eq). The reaction mixture was heated to reflux for 24 hours. The mixture was cooled to rt. Water (3 times the volume of DMSO) was then added. The mixture was extracted with EtOAc for 3 times. The combined organic phase was then concentrated. Purification by column chromatography (hexanes:EtOAc=8:1 to 3:1) gave the NON-S56 ligand.

### Synthesis of PtNON-S56

To a solution of NON-S56 ligand (1 eq) in HOAc (0.02 M) were added K₂PtCl₄ (1.1 eq) and n-Bu₄NBr (0.1 eq). The mixture was heated to reflux for 3 days. The reaction mixture was cooled to room temperature and filtered through a short pad of silica gel. The filtrate was concentrated under reduced pressure. Purification by column chromatography (hexanes:DCM=1:2 to 1:3) gave the PtNON-S56 in 59% yield.

General Procedure for Pt Complexes with General Formula IX

To a solution of corresponding ligand (1 eq) in 2-ethoxyethanol (0.02 M) were added K₂PtCl₄ (1.1 eq) and n-Bu₄NBr (0.1 eq). The mixture was heated to reflux for 3 days. The reaction mixture was cooled to room temperature and filtered through a short pad of silica gel. The filtrate was concentrated under reduced pressure. Purification by column chromatography (hexanes:DCM) gave the corresponding complexes.

### Example 1. Synthetic Route for Pt3O3

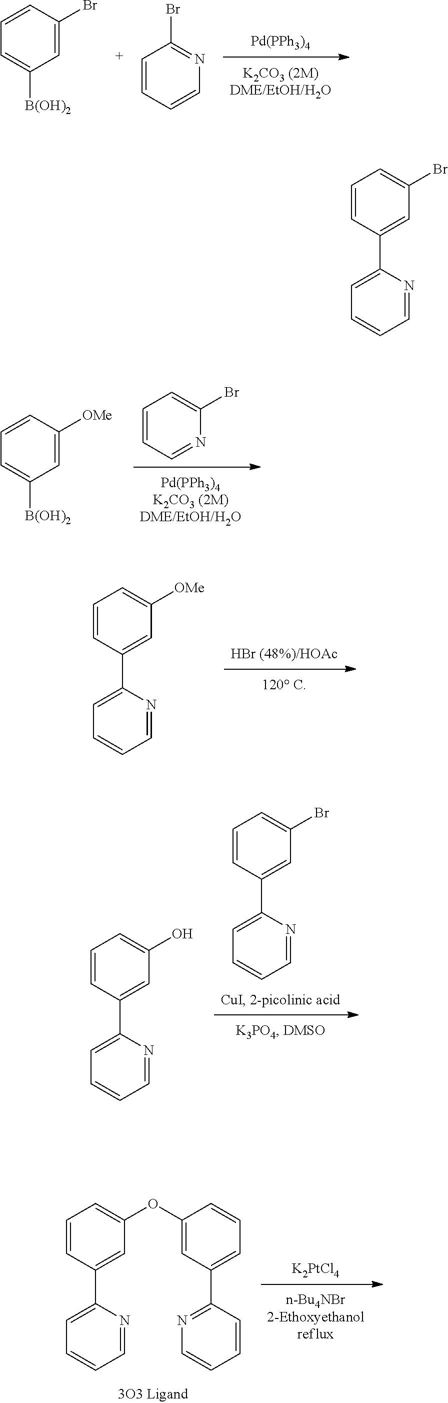

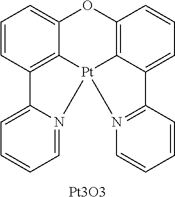

### Synthesis of 2-(3-bromophenyl)pyridine

To an oven-dried flask were added (3-bromophenyl)boronic acid (1.1 eq), 2-bromopyridine (1 eq), Pd(PPh₃)₄ (0.1 eq), EtOH/H₂O (3:3:1 ratio, 0.2 M for EtOH) and aqueous K₂CO₃ (2 M, 10 eq). The mixture was heated in an 80° C. oil bath for 24 hours. The mixture was cooled to room temperature. The solvent was then removed under reduced pressure. The product was isolated by column chromatography (Hexane:EtOAc=15:1 to 8:1) in 88% yield.

### Synthesis of 2-(3-methoxyphenyl)pyridine

To an oven-dried flask were added (3-methoxyphenyl)boronic acid (1.1 eq), 2-bromopyridine (1 eq), Pd(PPh₃)₄ (0.1 eq), EtOH/H₂O (3:3:1 ratio, 0.2 M for EtOH) and aqueous K₂CO₃ (2 M, 10 eq). The mixture was heated in an 80° C. oil bath for 24 hours. The mixture was cooled to room temperature. The solvent was then removed under reduced pressure. The product was isolated by column chromatography (Hexane:EtOAc=10:1 to 6:1) in 93% yield.

### Synthesis of 3-(pyridin-2-yl)phenol

To a solution of 2-(3-methoxyphenyl)pyridine (1 eq) in acetic acid (0.1 M) was added aqueous HBr (48 wt %, 10 eq). The mixture was heated in a 120° C. oil bath for 12 hours. The mixture was cooled to room temperature and water (equal volume to acetic acid) was then added. The mixture was neutralized with solid K₂CO₃ to pH 5-6. The precipitate was collected by filtration, rinsed with water 3 times, and dried under reduced pressure. The yield was quantitative.

### Synthesis of 3O3 Ligand

The reaction mixture was heated to reflux for 24 hours. The mixture was cooled to room temperature. Water (3 times the volume of DMSO) was then added. The mixture was extracted with EtOAc 3 times. The combined organic phase was then concentrated. Purification by column chromatography (hexanes:EtOAc=8:1 to 3:1) gave the 3O3 ligand in 85% yield.

### Synthesis of Pt3O3

To a solution of 3O3 ligand (1 eq) in EtOCH₂CH₂OH (0.02 M) were added K₂PtCl₄ (1.1 eq) and n-Bu₄NBr (0.1 eq). The mixture was heated to reflux for 3 days. The reaction mixture was cooled to room temperature and the solvent was removed under reduced pressure. Water was then added and the precipitate was then filtered and rinsed with water 3 times. The collected precipitate was then dried to give the crude product in quantitative yield.

### Example 2. Synthetic Route to Pt3O3-dtb

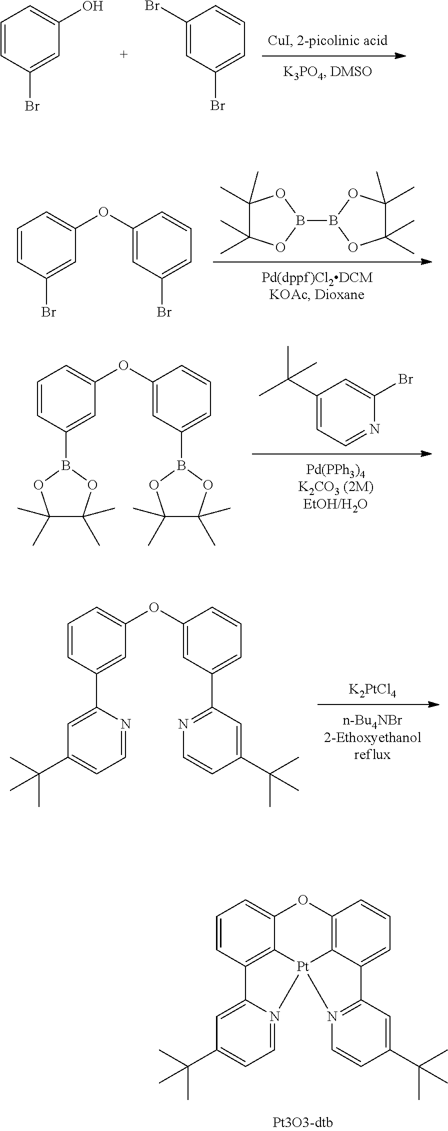

### Synthesis of 3,3′-oxybis(bromobenzene)

To a solution of 3-bromophenol (1 eq) in DMSO (0.5 M) were added CuI (0.1 eq), 2-picolinic acid (0.2 eq), K₃PO₄ (2 eq) and 1,3-dibromobenzene (3 eq). The mixture was heated with 100° C. oil bath for 24 hours. The mixture was cooled to room temperature. Water (3 times the volume of DMSO) was then added. The mixture was extracted with EtOAc for 3 times. The combined organic phase was then concentrated. Purification by column chromatography (hexanes) gave the product in 55% yield.

### Synthesis of 2,2′-(oxybis(3,1-phenylene))bis(4,4,5,5-tetramethyl-1,3,2-dioxaborolane)

To a solution of 3,3′-oxybis(bromobenzene) (1 eq) in dioxane (0.2 M) were added 4,4,4′,4′,5,5,5′,5′-octamethyl-2,2′-bi(1,3,2-dioxaborolane) (3 eq), Pd(dppf)Cl₂·DCM (0.1 eq) and KOAc (4 eq). The mixture was heated with 80° C. oil bath for 24 hours. The mixture was cooled to room temperature. The solvent was then removed under reduced pressure. The product was isolated by column chromatography (Hexane:EtOAc=50:1 to 10:1) in 70% yield.

### Synthesis of 3O3-Dtb Ligand

To an oven-dried flask were added 2,2′-(oxybis(3,1-phenylene))bis(4,4,5,5-tetramethyl-1,3,2-dioxaborolane) (1 eq), 2-bromo-4-(tert-butyl)pyridine (2.4 eq), Pd(PPh₃)₄ (0.1 eq), EtOH/H₂O (2:1 ratio, 0.2 M) and aqueous K₂CO₃ (2M, 10 eq). The mixture was heated with 80° C. oil bath for 24 hours. The mixture was cooled to room temperature. The solvent was then removed under reduced pressure. The product was isolated by column chromatography (Hexane:EtOAc=10:1 to 4:1) in 64% yield.

### Synthesis of Pt3O3-dtb

To a solution of 3O3-dtb ligand (1 eq) in HOAc (0.02 M) were added K₂PtCl₄ (1.1 eq) and n-Bu₄NBr (0.1 eq). The mixture was heated to reflux for 3 days. The reaction mixture was cooled to room temperature and filtered through a short pad of silica gel. The filtrate was concentrated under reduced pressure. Purification by column chromatography (hexanes:DCM=1:2 to 1:3) gave the Pt3O3-dtb in 68% yield.

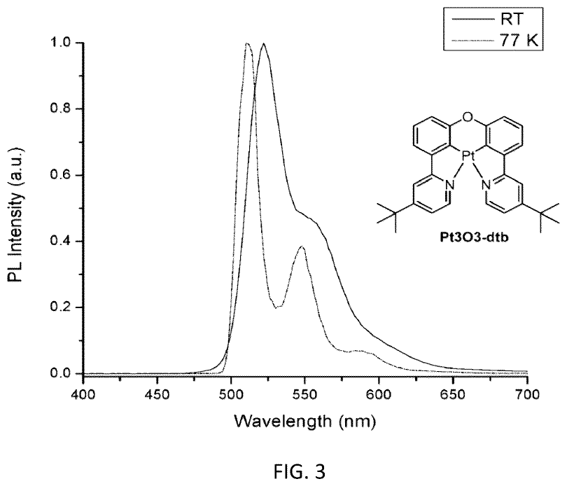

FIG. 3 shows emission spectra of Pt3O3-dtb in methylene chloride at 77K and room temperature.

1.-10. (canceled)

11. A complex represented by General Formula X: 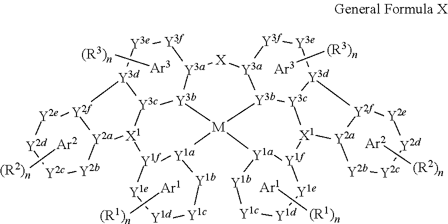
wherein:
the complex has an axis of symmetry through M and X;
M is Pt²⁺;
R¹, R², R³, and R⁴, if present each independently represents hydrogen, deuterium, trideuteriummethyl, pentadeuteriumphenyl, halogen, hydroxy, nitro, nitrile, thiol; or substituted or unsubstituted, amino, alkoxy, aryl, or C₁-C₄ alkyl;
two adjacent groups R¹, two adjacent groups R², or two adjacent groups R³ together form a group of Formula A 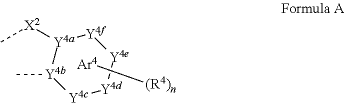
each n independently represents an integer, valency permitting;
Y^{1a}, Y^{1b}, Y^{1c}, Y^{1d}, Y^{1e}, Y^{1f}, Y^{2a}, Y^{2b}, Y^{2c}, Y^{2d}, Y^{2e}, Y^{2f}, Y^{3a}, Y^{3b}, Y^{3c}, Y^{3d}, Y^{3e}, Y^{3f}, Y^{4a}, Y^{4b}, Y^{4c}, Y^{4d}, Y^{4e}, and Y^{4f} each independently represents C, N, Si, O, or S; and
each of X, X¹ and X² is present or absent, and each X, X¹, and X² present independently represents a single bond, NR, PR, CRR′, SiRR′, CRR′, SiRR′, O, S, S═O, O═S═O, Se, Se═O, or O═Se═O, and wherein R and R′ each independently represents hydrogen, nitrile, halogen, hydroxy, nitro, thiol, or substituted or unsubstituted alkoxy, aryl, amino, or C₁-C₄ alkyl.

12. The complex of claim 11, wherein Ar¹, Ar², Ar³, and Ar⁴ independently represent aryl or heteroaryl groups.

13. The complex of claim 11, wherein two adjacent groups R¹ represent a group of Formula A.

14. The complex of claim 11, wherein two adjacent groups R² represent a group of Formula A.

15. The complex of claim 14, wherein X² is bound to Y^{2c}; and wherein Y^{4b} is bound to Y^{2b}.

16. The complex of claim 14, wherein X² is bound to Y^{2b}; and wherein Y^{4b} is bound to Y^{2e}.

17. The complex of claim 11, wherein two adjacent groups R³ represent a group of Formula A.

18. The complex of claim 11, wherein X is O.

19. The complex of claim 11, wherein X¹ is N.

20. The complex of claim 11, wherein X² is O.

21. The complex of claim 11, wherein X² is S.

22. The complex of claim 11, wherein the complex is represented by General Formula III:

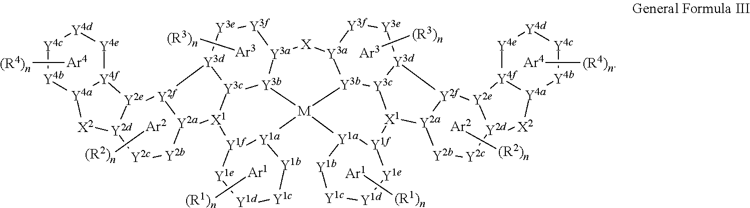

23. The complex of claim 11, wherein the complex is represented by General Formula IV:

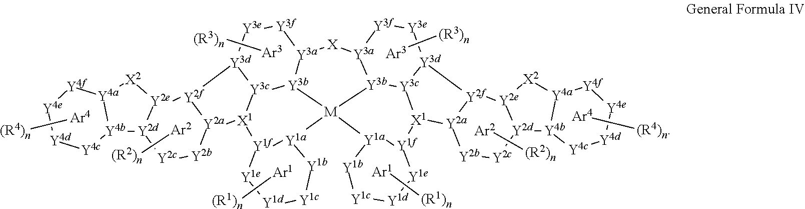

24. The complex of claim 11, wherein the compound is represented by General Formula V:

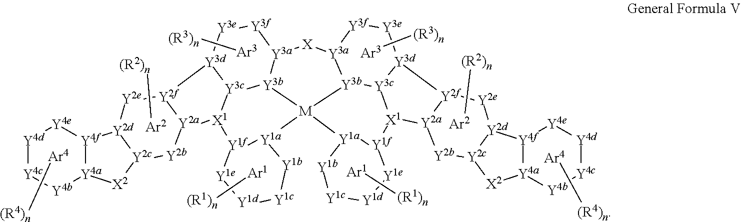

25. The complex of claim 11, wherein the compound is represented by General Formula VI:

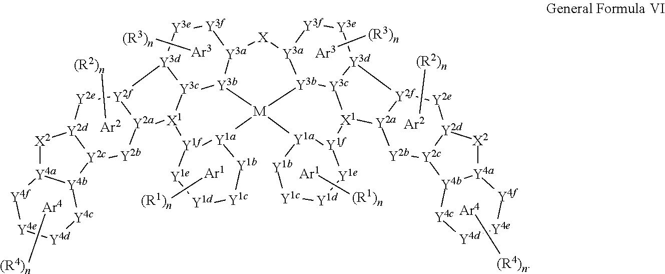

26. The complex of claim 11, wherein the complex is represented by General Formula VII:

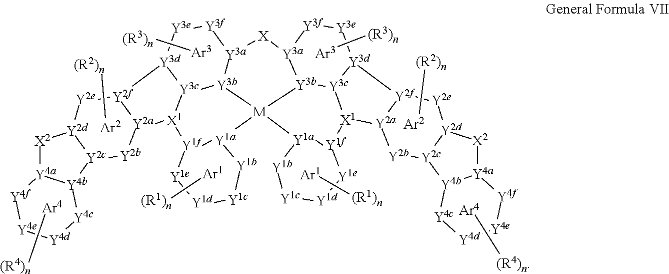

27. The complex of claim 11, wherein the complex is represented by General Formula VIII:

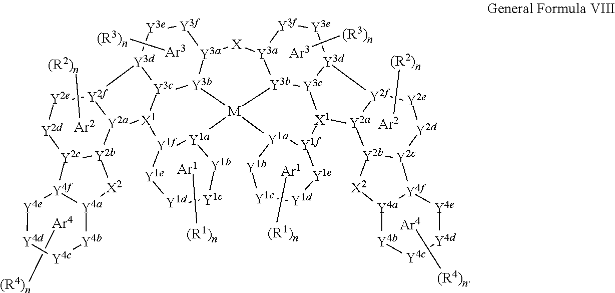

28. A complex represented by one of the following structures:

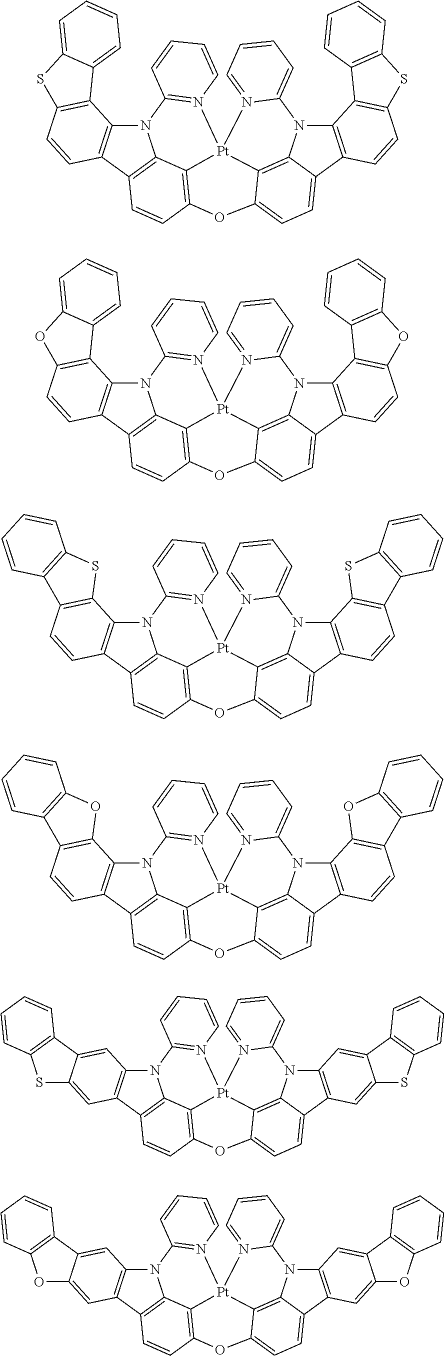

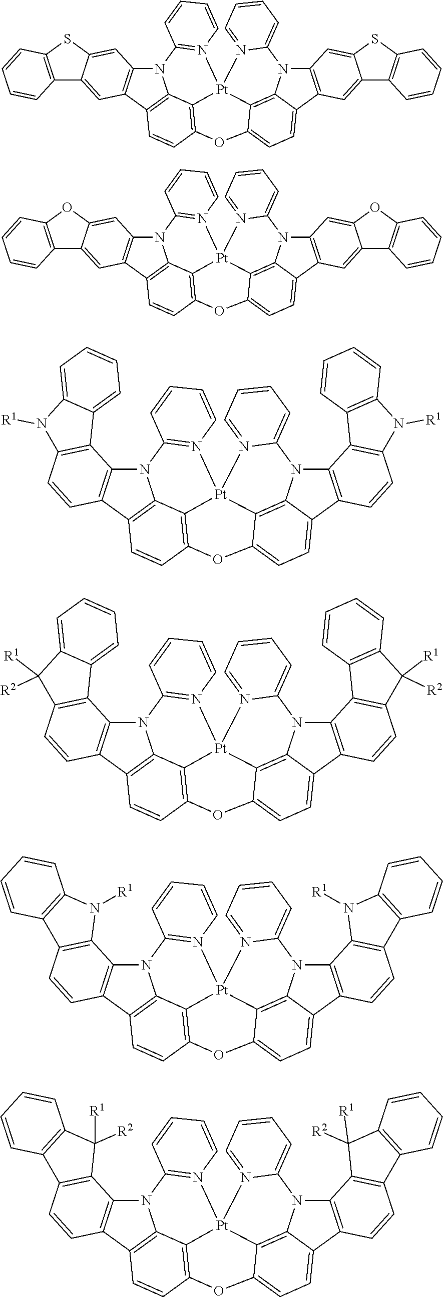

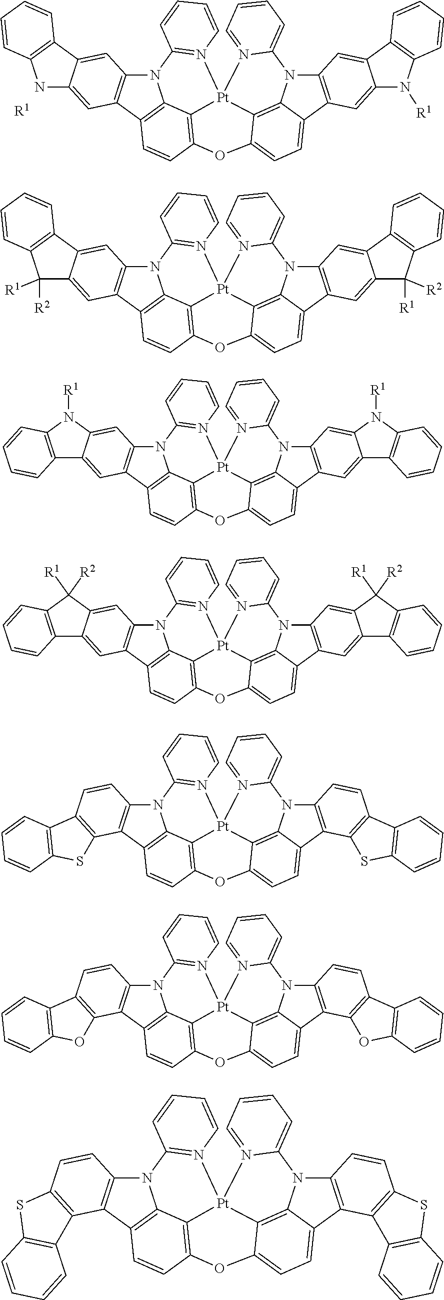

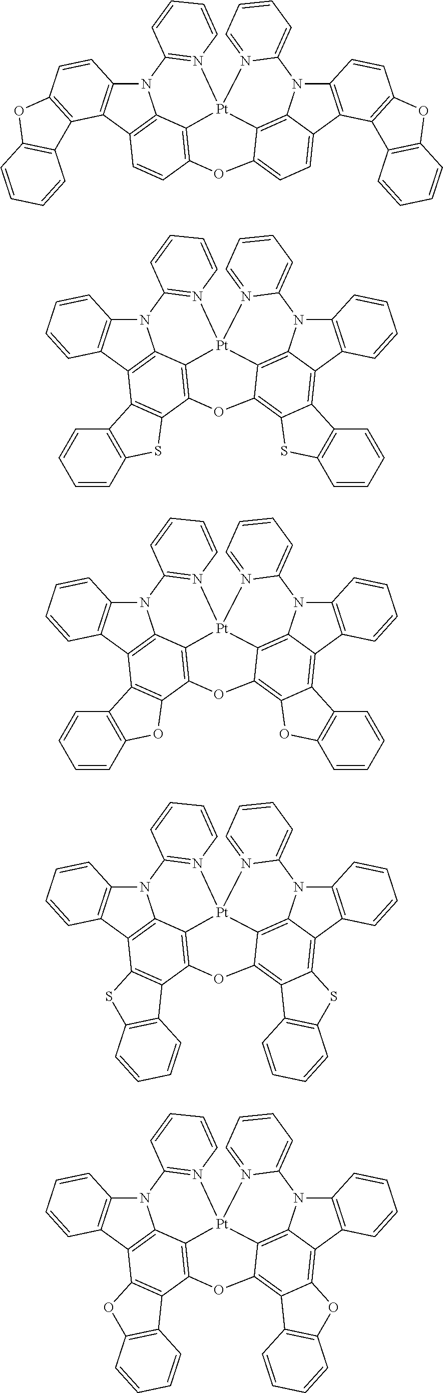

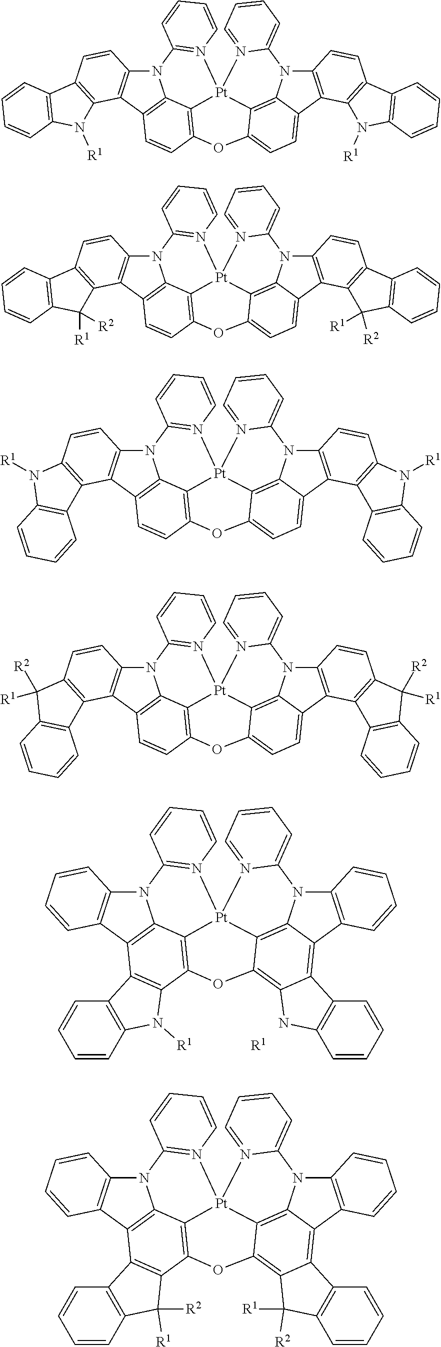

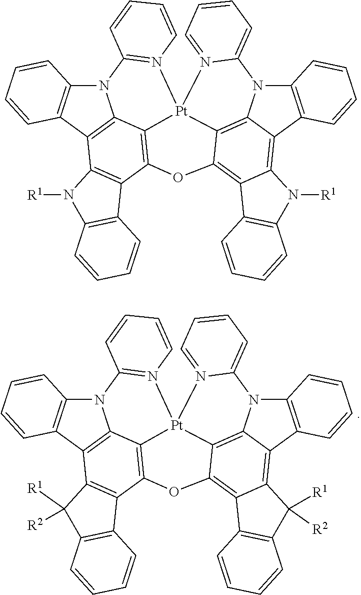

29. An organic light-emitting device comprising the complex of claim 11.

30. A light-emitting device comprising the compound of claim 28.
